(19) United States
(12) Patent Application Publication
(10) Pub. No.: US 2023/0169648 A1
(43) Pub. Date: Jun. 1, 2023

(54) System, Control Method, Information Providing Method, and Recording Medium

(71) Applicant: Splink, Inc., Tokyo (JP)

(72) Inventors: Wataru Kasai, Tokyo (JP); Akihiro Okuno, Tokyo (JP); Hirofumi Shido, Tokyo (JP)

(21) Appl. No.: 17/921,775

(22) PCT Filed: Feb. 24, 2021
(86) PCT No.: PCT/JP2021/006756

§ 371 Date: Oct. 27, 2022

(30) Foreign Application Priority Data
Apr. 28, 2020 (JP) 2020-079504

(51) Int. Cl. G06T 7/00; A61B 5/00

(52) U.S. Cl. CPC G06T 7/0012; A61B 5/4064; A61B 5/4088; G06T 2207/30016

(57) ABSTRACT

There is provided a system for determining the state of dementia by using an image differentiation technique and a cognitive test score together with each other.

A system includes: a first input module configured to acquire a first evaluation index based on data regarding the physical state of a brain of a subject; a second input module configured to acquire a second evaluation index based on data regarding the function of the brain of the subject; and an estimation module configured to estimate the state of dementia of the subject based on an evaluation value obtained by a first evaluation function having the first evaluation index and the second evaluation index as its variables.

### CROSS-REFERENCE TO RELATED APPLICATIONS

This application is the national phase under 35 U.S.C. § 371 of PCT International Application No. PCT/JP2021/006756 which has an International filing date of Feb. 24, 2021 and designated the United States of America.

### FIELD

The present disclosure relates to a system capable of evaluating dementia.

### BACKGROUND

Japanese Patent Laid-Open Publication No. 2011-206452 describes providing a driving aptitude diagnosis device and a driving aptitude diagnosis method capable of diagnosing the driving aptitude of a subject with high reliability without being affected by the examination environment, the physical or mental condition of a subject, the arbitrariness of an examiner, and the like.

The driving aptitude diagnosis device includes a white matter lesion examination means for examining the degree of a cerebral white matter lesion in a subject and a driving aptitude determination means for determining the driving aptitude of the subject based on the examination result of the white matter lesion examination means. The driving aptitude determination means determines that the subject's driving aptitude is not suitable when the degree of the white matter lesion examined by the white matter lesion examination means is equal to or greater than a specified value.

### SUMMARY

Matsuda, Hiroshi et al., “Differentiation Between Dementia With Lewy Bodies And Alzheimer's Disease Using Voxel-Based Morphometry Of Structural MRI: A Multicenter Study.” (Neuropsychiatric Disease and Treatment 15 (2019): 2715.) discloses a technique for image differentiation between Alzheimer's disease and dementia with Lewy bodies using brain images.

Shimomura, Tatsuo et al., “Cognitive loss in dementia with Lewy bodies and Alzheimer disease.” (Archives of Neurology 55.12 (1998): 1547-1552.) discloses that there is a bias in the distribution of specific cognitive test scores (WAIS-R Block Design test score or ADAS delayed recall score) in Alzheimer's disease and dementia with Lewy bodies.

However, there is no disclosure of a technique for determining the state of dementia by using the image differentiation technique and the cognitive test score together with each other.

One aspect of the present disclosure is a system including: a first input module configured to acquire a first evaluation index based on data regarding a physical state of a brain of a subject; a second input module configured to acquire a second evaluation index based on data regarding a function of the brain of the subject; and an estimation module configured to estimate a state of dementia and/or other brain disorders of the subject based on an evaluation value obtained by a first evaluation function having the first evaluation index and the second evaluation index as its variables. The evaluation result of data regarding the physical state of the brain and the evaluation result of data regarding the function of the brain can be convoluted into one collaborative evaluation result by using the evaluation function, so that it is possible to evaluate the state of dementia with higher accuracy. Therefore, it is possible to provide a system for determining an affection state including the presence or absence of a brain disease of the subject or for supporting the determination. In addition, when the subject is included in a group ingesting drugs (including investigational drugs and unapproved drugs), food and drink, and supplements, the estimation module may have a function (unit) of evaluating their effects on dementia. In addition, the estimation module may have a function of estimating the affection state of a first causative disease.

Another aspect of the present disclosure is a method for controlling a system. The system includes: a first input module configured to acquire a first evaluation index based on data regarding a physical state of a brain of a subject, a second input module configured to acquire a second evaluation index based on data regarding a function of the brain of the subject, and an estimation module configured to estimate a state of a brain disorder, including dementia, of the subject. The method includes the following steps.

1. Acquiring the first evaluation index and the second evaluation index through the first input module and the second input module by the estimation module.

2. Estimating a state of dementia of the subject based on an evaluation value obtained by a first evaluation function having the first evaluation index and the second evaluation index as its variables. The estimated state of dementia may include at least one of pieces of information required for prevention and treatment of dementia, such as the presence or absence of dementia, stage, and causative disease.

Still another aspect of the present disclosure is a computer readable non-transitory recording medium recording a program. A program (program product) recorded in a recording medium has instructions for causing a computer to execute: acquiring a first evaluation index based on data regarding a physical state of a brain of a subject; acquiring a second evaluation index based on data regarding a function of the brain of the subject; and estimating a state of dementia of the subject based on an evaluation value obtained by a first evaluation function having the first evaluation index and the second evaluation index as its variables. The program may be provided in a state in which the program is recorded on a computer-readable recording medium.

The above and further objects and features of the invention will more fully be apparent from the following detailed description with accompanying drawings.

### BRIEF DESCRIPTION OF THE DRAWINGS

FIG. 1 is a block diagram showing the outline of a dementia evaluation system.

FIG. 2 is a flowchart showing a dementia evaluation method.

FIG. 3 is a diagram showing an example of an examination or test for measuring brain health.

FIG. 4 is a diagram, subsequent to FIG. 3, showing an example of an examination or test for measuring brain health.

FIG. 5 is a score example of a cognitive test.

FIG. 6 is an example of an odds table.

FIG. 7 is a diagram showing the result of evaluation by odds.

FIG. 8 is a diagram showing the result of logistic regression evaluation.

FIG. 9 is a diagram showing the result of evaluation using the Z-score.

FIG. 10 is a diagram showing categorization.

FIG. 11 is a schematic diagram showing a first example of the configuration of a system according to a third embodiment.

FIG. 12 is a schematic diagram showing an example of the evaluation result of a hippocampal region.

FIG. 13 is a schematic diagram showing an example of the evaluation result of a middle temporal gyrus region.

FIG. 14 is a schematic diagram showing an example of the evaluation value of each part.

FIG. 15 is a schematic diagram showing a first example of a part comparison screen.

FIG. 16 is a schematic diagram showing a second example of the part comparison screen.

FIG. 17 is a schematic diagram showing a third example of the part comparison screen.

FIG. 18 is a schematic diagram showing an example of Z-score correlation between each region of the left brain and each region of the right brain.

FIG. 19 is a schematic diagram showing an example of a left-brain and right-brain aggregation method.

FIG. 20 is a schematic diagram showing a specific example of a whole-brain evaluation value.

FIG. 21 is a schematic diagram showing a first example of a whole-brain evaluation screen.

FIG. 22 is a schematic diagram showing a second example of the whole-brain evaluation screen.

FIG. 23 is a schematic diagram showing a third example of the whole-brain evaluation screen.

FIG. 24 is a schematic diagram showing a fourth example of the whole-brain evaluation screen.

FIG. 25 is a schematic diagram showing a fifth example of the whole-brain evaluation screen.

FIG. 26 is a schematic diagram showing a second example of the configuration of the system according to the third embodiment.

FIG. 27A is a schematic diagram showing an example of an ROC curve.

FIG. 27B is a schematic diagram showing an example of an ROC curve.

FIG. 28 is a flowchart showing the procedure of a first example of a process for outputting an evaluation result of each part of the brain.

FIG. 29 is a flowchart showing the procedure of a second example of a process for outputting an evaluation result of each part of the brain.

FIG. 30 is a flowchart showing the procedure of a process for outputting a whole-brain evaluation result.

### FIRST EMBODIMENT

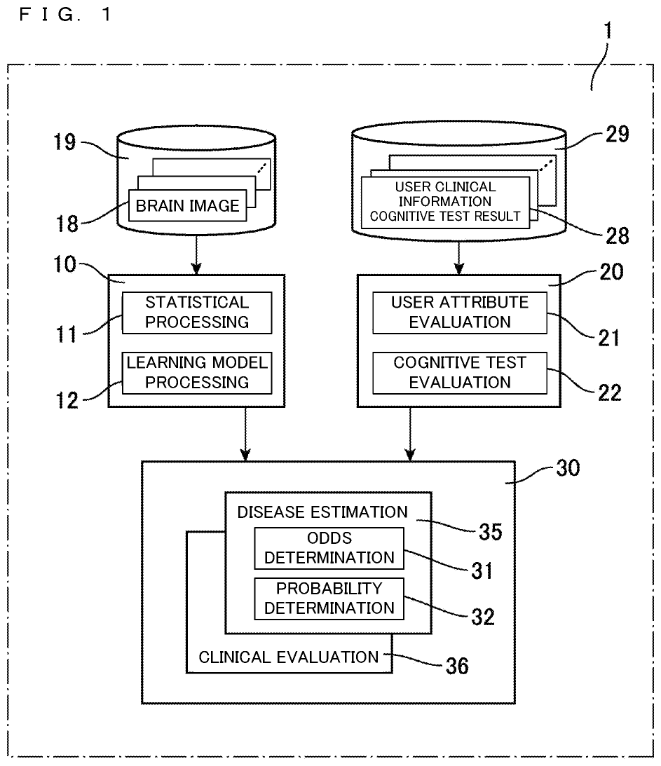

FIG. 1 shows a system (dementia evaluation system) 1 for evaluating brain disorders, such as dementia, according to the present disclosure. The system 1 includes: a first input module 10 configured to acquire a first evaluation index X1 based on data regarding the physical state of the brain of a subject; a second input module 20 configured to acquire a second evaluation index X2 based on data regarding the brain function of the subject; and an estimation module 30 configured to estimate the state of the subject's brain disorder including dementia (dementia and/or other brain disorders) based on an evaluation value fv obtained by a first evaluation function f1 having the first evaluation index X1 and the second evaluation index X2 as its variables. The system 1 further includes a database 19 in which image data including a brain image 18 of the subject is stored and a database 29 in which clinical information 28 including the cognitive test result of the subject is stored.

Brain disorders mainly include higher brain dysfunction such as dementia, attention disorders, memory disorders, executive function disorders, social behavior disorders, aphasia, apraxia, and agnosia. Dementia includes AD (Alzheimer Disease), DLB (Dementia with Lewy Bodies), and other kinds of degenerative dementia such as frontotemporal dementia, progressive supranuclear palsy, corticobasal degeneration, and argyrophilic grain dementia. The state of brain disorders includes various aspects relevant to the brain disorders of subjects (testees, patients, and users), such as the presence or absence of brain disorders, the progress of brain disorders, the presence or absence and differentiation of causative diseases of brain disorders such as dementia, and the progress of single or multiple causative diseases.

One of the purposes of the system 1 is to realize numerical quantification by combining a plurality of evaluations for various states of subject's dementia, brain disorders including dementia, and brain disorders not including dementia so that it is possible to categorize the various states and to provide information for further evaluation analysis for each category. It is effective not only in the treatment of subjects but also in other fields, such as clinical research, to divide subjects with various states of brain disorders into categories and perform evaluation analysis. For example, the system 1 is effective for evaluating the effects and influences of various items taken by people, such as drugs, food and drink, and supplements, on the brain or brain disorders including dementia, and may also be used as a stratification marker. In addition, the system 1 may also be applied to the evaluation of information devices, games, and other applications that may affect the brain or brain disorders. Examples of evaluating the state of dementia, including other purposes and advantages of the system, will be described below.

The first input module 10 includes: a first evaluation unit 11 that acquires the first evaluation index X1 by statistically evaluating a first type medical image of a region of interest of at least a part of the subject's brain; and a second evaluation unit 12 that acquires the first evaluation index X1 by evaluating a medical image of the subject by using a first model machine-learned to evaluate a first disease based on the first type medical image.

As a device for diagnosing the morphology and function of a test target (subject), various types of tomography devices (modalities) such as CT (Computed Tomography), MRI (Magnetic Resonance Imaging), PET (Positron Emission Tomography), SPECT (Single Photon Emission Computed Tomography), and PET-CT are known, and their modality images (medical images) are used for diagnosing various diseases. In particular, the modality image (medical image) 18 of the subject's brain is used to acquire data regarding the physical state of the subject's brain, and is used for diagnosing diseases, such as dementia and Parkinson's disease. In this specification, the physical state of the brain refers to a state in which the brain can be measured by a physical method, and indicates a state in which evaluation, measurement, and estimation can be performed by methods such as statistical processing and learning models based on various modality images including MRI, PET, and SPECT that can measure morphology, glucose metabolism, blood flow, and the like.

Examples of types of medical images are CT and MRI, and these images can reflect highly accurate morphological information. Other examples of types of medical imaging are PET and SPECT, and these images are generated by administering a radiopharmaceutical into the body of the subject using intravenous injection and imaging the radiation emitted from the drug in the body. According to images using drugs, doctors can see not only the morphology of each part in the body but also how the administered drug is distributed in the body and the state of accumulation of substances in the body that react with the drug. This can contribute to improving the diagnostic accuracy of diseases. For example, capturing a PET image using a so-called Pittsburgh compound B as a radiopharmaceutical (tracer) for PET and measuring the degree of accumulation of amyloid β protein in the brain based on the captured PET image can be helpful for differential diagnosis or early diagnosis of Alzheimer's dementia.

An example of a SPECT image is an imaging method for visualizing the distribution of a dopamine transporter (DAT) called DatSCAN (Dopamine transporter SCAN) in a SPECT examination in which a radiopharmaceutical called 123I-Ioflupane is administered. As purposes of this imaging, early diagnosis of Parkinson's syndrome (PS) of Parkinson's disease (hereinafter, PD), diagnostic aid for dementia with Lewy bodies (DLB), medication treatment determination called Levodova when there is striatal dopaminergic loss, and the like can be mentioned.

The first evaluation unit 11 uses statistical processing for the evaluation of medical images. The first evaluation unit 11 may present evaluation by performing a statistical comparison between the brain images of the subject and the brain images of healthy persons. As a method for evaluating brain atrophy using brain images, VBM (Voxel Based Morphometry) is known in which image processing on brain images acquired by imaging the subject's head is performed in units of voxels, which are three-dimensional pixels. A typical example of statistical processing is to generate a Z-score map. Therefore, the first evaluation unit 11 may acquire the Z-score as the first evaluation index X1.

Taking an MR image as an example, the Z-score is created by substituting the value of data (normal standard brain), from which an average image and a standard deviation image are created by calculating a mean value and a standard deviation for each voxel from MR images of normal cases subjected to brain morphology standardization processing, and the value of image data (processed image) of the subject into the following equation for calculating the Z-score.

z=(M(x,y,z)−I(x,y,z))/SD(x,y,z)

M and SD indicate an average image and a standard deviation image of a normal standard brain, respectively, and I indicates a processed image. By using the Z-score map, it is possible to quantitatively analyze what kind of change occurs in which part of the processed image compared with normal standard brains. For example, a voxel with a positive value in the Z-score map indicates a region with atrophy compared with normal standard brains, and the larger value can be interpreted as statistically greater divergence. For example, if the Z-score is “2”, this means that the value deviates from the mean value by twice the standard deviation, and it is evaluated that there is a statistically significant difference with a risk rate of about 5%. In order to quantitatively evaluate atrophy in a region, M, SD, and I may be calculated in the region of interest, and the average of all positive Z-scores may be calculated.

As examples of statistical processing, various methods, such as a method of comparing the volume or area of each part of the brain and a method of using a T-test using a general linear model (GLM), have been proposed.

Measuring the degree of accumulation of amyloid β protein in the brain based on the captured PET image by using a so-called Pittsburgh compound B as a radiopharmaceutical (tracer) for PET can be helpful for differential diagnosis or early diagnosis of Alzheimer's dementia. In the PET image, an SUVR (Standardized Uptake Value Ratio, cerebellar ratio SUVR) indicating the ratio between the sum of an SUV (Standardized Uptake Value) of the amyloid β protein in the cerebral gray matter of a part of the brain and the SUV of the amyloid β protein in the cerebellum can be adopted as statistical processing. SUVR can be defined by the following equation.

$$\begin{matrix}[ Equation 1 ] & \\ SUVR = \frac{\begin{matrix}SUV ( frontal ) + SUV ( cingulate ) + \\ SUV ( parietal ) + SUV ( temporal )\end{matrix}}{SUV ( cerebellum )} & \end{matrix}$$

The numerator of this equation indicates the sum of the SUVs of the four parts of cerebral gray matter, that is, the cortical regions (prefrontal cortex, posterior cingulate cortex, parietal lobe, and lateral temporal lobe) of the cerebrum, and the denominator indicates the SUV of the cerebellum.

In the statistical processing of DatSCAN using SPECT images, BR (Binding Ratio) can be adopted as an evaluation (index value), and is expressed by the following equation.

$$\begin{matrix}[ Equation 2 ] & \\ BR = \frac{{C}_{specific} - {C}_{nonspecific}}{{C}_{nonspecific}} & \end{matrix}$$

C in the equation indicates the mean value of DAT in each region of interest, Cspecific indicates the mean value of the putamen and caudate nucleus in the brain, and Cnonspecific indicates the mean value of the occipital cortex in the brain.

The second evaluation unit 12 evaluates the brain image 18 of the subject by using the first model (learning model) machine-learned to evaluate the first disease, for example, AD (Alzheimer's Disease) or DLB (Dementia with Lewy Bodies), based on medical images of a type common to or different from that of the first evaluation unit 11.

By using the model (learning model) machine-learned based on medical image information, differentiation of a disease from the medical images of the subject is performed. Iizuka, Tomomichi et al., “Deep-learning-based imaging-classification identified cingulate island sign in dementia with Lewy bodies.” (Scientific reports 9.1 (2019): 1-9.) reported that an experiment using a convolutional neural network for perfusion SPECT images achieved an even higher accuracy of 89.32% and deep learning focused on blood flow findings in the occipital lobe, which was used for interpretation in the past, in the differentiation. Litjens, Geert et al., “A survey on deep learning in medical image analysis.” (Medical image analysis 42 (2017): 60-88.) and Wen, Junhao et al., “Convolutional Neural Networks for Classification of Alzheimer's Disease: Overview and Reproducible Evaluation.” (CoRR abs/1904.07773 (2019)) reported that the application of recent deep learning technology showed high accuracy in the differentiation of AD.

The second evaluation unit 12 may acquire, as the first evaluation index X1, an output softmax value Xa of the activation function when estimating the first causative disease using the deep learning differentiation model. The first input module 10 may output, as the first evaluation index X1, the following values obtained by the first evaluation unit 11 and/or the second evaluation unit 12.

Xa: output softmax value of an activation function when estimating the first causative disease by the deep learning differentiation model using a brain image as its input.

Xb: output softmax value of an activation function when estimating the first causative disease by the deep learning differentiation model further using a filtered image of a brain image as its input in a region of interest obtained by statistical processing of brain images.

Xc: volume value or blood flow rate of a region of interest by statistical processing of brain images.

Xd: Z-score value of volume or blood flow evaluation of a region of interest by statistical processing of brain images.

Xe: volume value or blood flow rate of a region of interest when estimating the first causative disease by the deep learning differentiation model using a brain image as its input.

Xf: Z-score value of the volume or blood flow evaluation of a region of interest when estimating the first causative disease by the deep learning differentiation model using a brain image as its input.

The second input module 20 includes a configuration for acquiring an evaluation of the clinical information 28 including a cognitive test as the second evaluation index X2. The second input module 20 includes a unit 22 that evaluates a cognitive test and a unit 21 that evaluates other pieces of clinical information regarding user attributes.

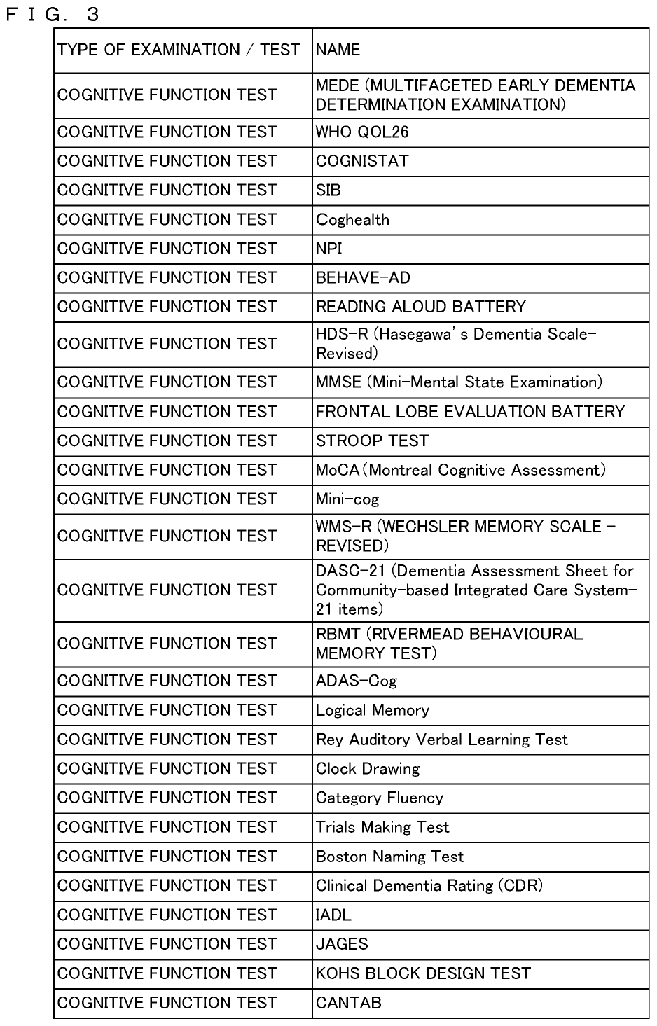

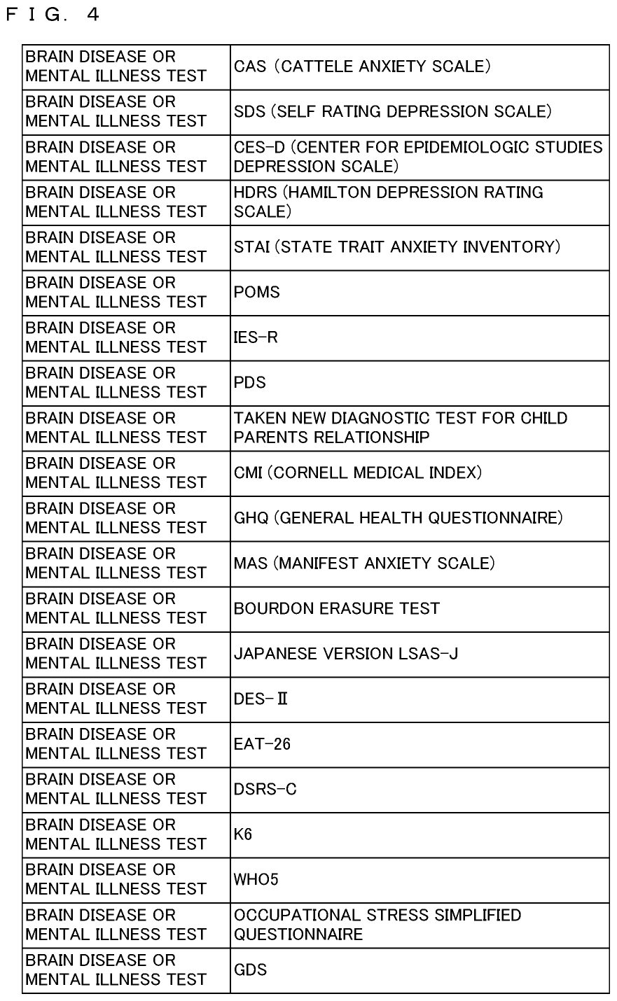

The cognitive test is used as a means for acquiring data regarding the function of the brain of the subject, particularly as a test for checking the cognitive function of the brain. Contents of the cognitive test include, but are not limited to, calculations such as addition and subtraction, Stroop, N-Back, and fast writing of words. Specific examples of the cognitive test are disclosed in, for example, Japanese Patent Laid-Open Publication No. 2019-75071, and include tests for “Digit Span Forward” and “Digit Span Backward”, tests for “Stroop”, tests for “addition” and “subtraction”, tests for N-Back (for example, 1-Back), tests for “immediate recall” (word recall). In addition, the cognitive test is not limited to these, and examinations or tests for measuring brain health (including the state of cognitive function and the presence or absence and degree of brain diseases and mental disorders) listed in FIGS. 3 and 4 and similar forms of examinations or tests may be adopted.

By combining these tests arbitrarily or by combining all of the tests, it is possible to provide a cognitive test suitable for determining the overall picture of a brain function or the affection state of a specific causative disease. In this specification, the “brain function” refers to the ability to make determinations based on artificial actions involving the brain, such as expression and comprehension, other than the physical state of the brain. Typically, the brain function may be appropriately determined according to the result of the appropriate cognitive test.

In the result of the cognitive test, the state of brain function can be scored based on, for example, the reaction time (response time) of the subject with respect to the cognitive test or the number of correct answers (hereinafter, this score is referred to as a “cognitive score”). Cognitive score information may be used as the second evaluation index X2 for evaluating the subject's brain function. By expressing the result of the cognitive test (cognitive score) as a normal distribution, it is possible to estimate brain age. The evaluation index may be corrected by using the age of the subject. The evaluation index for evaluating the state of brain function can be calculated based on clinical information including the cognitive test score. The clinical information may include age, sex, education history, work history, genes (ApoE, and the like), blood test results, interview results (ADL interview, and the like) in addition to the cognitive test score.

The second evaluation index X2 based on the data regarding the brain function is also effective as an index that accurately indicates the estimated range of dementia risk from pre-MCI (preclinical stage of Mild Cognitive Impairment) to MCI (Mild Cognitive Impairment, mild dementia), then to AD (Alzheimer Disease).

A typical example of the estimation module 30 that estimates the state of dementia of the subject based on the evaluation value fv obtained by the first evaluation function f1 having the above-described first evaluation index X1 and second evaluation index X2 as its variables is a function (disease estimation function, unit) 35 for estimating the affection state of the first causative disease, for example, AD or DLB. The first input module 10 includes a configuration for acquiring the first evaluation index X1 for differentiation of the first causative disease, and the second input module 20 includes a configuration for acquiring the second evaluation index X2 for differentiation of the first causative disease. The second input module 20 may include a configuration for acquiring the second evaluation index X2 including a result 28 of a cognitive test suitable for differentiation of the first causative disease.

Another example of the estimation module 30 is a function (clinical evaluation function, clinical evaluation unit) 36 for evaluating the effect of ingesta on dementia when the subject is included in a group ingesting at least one of drugs, food and drink, and supplements. The evaluation result in the evaluation function 36 can be used as a stratification marker corresponding to a biomarker in stratified medicine. Therefore, the system 1 may include a module that provide information as a stratification marker based on the estimation of the estimation module 30. In the system 1, as will be described in more detail below, it is possible to provide stratification markers quantified by the cooperation of two or more factors, classify patients into categories according to purposes using quantified markers, and find categories necessary for evaluation for research, medical care, and the like. Hereinafter, the unit 35 for estimating the affection state will be further described as an example.

The affection estimation unit 35 includes an odds determination unit 31 that determines the presence or absence of affection based on odds and a probability determination unit 32 that determines the probability of affection. The odds determination unit 31 includes a configuration for calculating an evaluation value s by the following first evaluation function fla in which the first evaluation index X1 is the odds x1 of the first causative disease and the second evaluation index X2 is the odds x2 of the first causative disease.

s=x1×x2 (f1a)

Assuming that the first evaluation index X1 and the second evaluation index X2 are xi and the weighting factor of each value is wi, the probability determination unit 32 includes a configuration for calculating an affection probability p as an evaluation value by using the following first evaluation function f1b.

$$\begin{matrix}[ Equation 3 ] & \\ p = \frac{1}{1 + exp ( - {{\sum }_{i = 0}}^{n} {w}_{i} {x}_{i} )} & ( f 1 b )\end{matrix}$$

Here, i is an integer. In addition, the first evaluation index X1 may be any one of the values Xa to Xf described above, or may include a plurality of values.

According to the logistic regression model, the logarithmic odds of the affection probability p of one A (AD or DLB) of two classes, that is, causative diseases are expressed by the following equation.

$$\begin{matrix}[ Equation 4 ] & \\ log ( \frac{p}{1 - p} ) = {w}_{0} {x}_{0} + {w}_{1} {x}_{1} + … + {w}_{n} {x}_{n} = {{\sum }_{i = 1}}^{N} {w}_{i} {x}_{i} & \end{matrix}$$

The coefficient wi can be calculated by maximum likelihood estimation (stochastic gradient descent method and the like).

According to the evaluation function f1b, it is possible to obtain the determination of disease A if the affection probability p is greater than 0.5 and the determination of no disease A if the affection probability p is equal to or less than 0.5. When evaluating a plurality of causative diseases (a plurality of classes), a plurality of sets of one vs all or one vs rest may be created, and it may be determined that the causative disease (class) with the maximum p value corresponds. Based on the multivalued logistic regression, a first evaluation function f1c of a desired causative disease may be expressed as follows.

$$\begin{matrix}[ Equation 5 ] & \\ {p}_{y *} = \frac{exp ( - {{\sum }_{i}}^{n} {w}_{y * i} {x}_{i} )}{{\sum }_{y ∈ Y} exp ( - {{\sum }_{i}}^{n} {w}_{yi} {x}_{i} )} & ( f 1 c )\end{matrix}$$

Here, y* is a target class (disease), Y is a set of all classes (diseases) to be evaluated, and wyi is a weighting factor for each evaluation index of class (disease) y.

Degenerative dementias include, as non-AD dementias, not only dementia with Lewy bodies but also frontotemporal dementia, progressive supranuclear palsy, corticobasal degeneration, and argyrophilic grain dementia, and the first evaluation function f1c can be used for differentiation of these causative diseases.

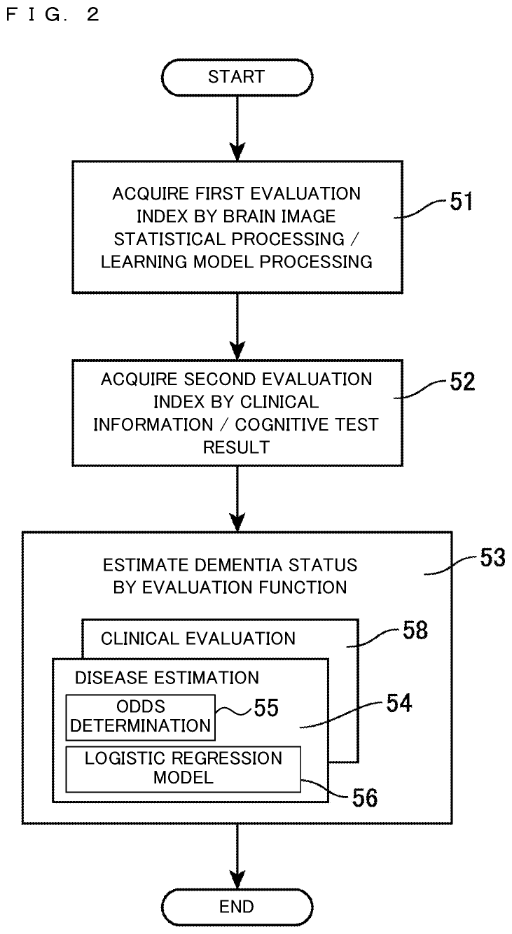

In FIG. 2, an evaluation method using the dementia evaluation system 1 is shown by the flowchart. The dementia evaluation system 1 can be provided as an information processing apparatus including computer resources including a memory and a CPU, and the control method can be provided as a program having executable instructions on a computer. The program (program product) may be recorded on a computer-readable recording medium and provided, or may be provided in a downloadable state from the Internet or the like. In addition, the dementia evaluation system 1 may be provided as a service (SaaS (Software as a Service)) through the Internet.

In the system 1 including the first input module 10, the second input module 20, and the estimation module 30 configured to estimate the dementia state of the subject, the estimation module 30 acquires the first evaluation index X1 from the first input module 10 in step 51, and the estimation module 30 acquires the second evaluation index X2 through the second input module 20 in step 52. In addition, in step 53, the estimation module 30 estimates the dementia state of the subject based on the evaluation value obtained by the first evaluation function having the first evaluation index X1 and the second evaluation index X2 as its variables, for example, the above-described evaluation function f1a or f1b.

In step 53, the estimation module 30 may perform a process 54 for estimating the affection state of the first causative disease. In addition, when the subject is included in a group ingesting at least one of drugs, food and drink, and supplements, the estimation module 30 may perform a process 58 for evaluating the effect of ingesta on dementia.

In step 51, the estimation module 30 may acquire a value obtained by statistically evaluating the medical image by the first evaluation unit 11, as the first evaluation index X1, through the first input module 10, may acquire a value obtained by evaluating the medical image by the second evaluation unit 12 as the first evaluation index X1, or may acquire the first evaluation index X1 reflecting both the statistically evaluated value of the medical image and the value when the medical image of the subject is evaluated by using the machine-learned model (first model) as indicated by the values Xa to Xf.

In addition, in step 52, the estimation module 30 may acquire an evaluation of clinical information including a cognitive test as the second evaluation index X2. In addition, in steps 51 and 52, the estimation module 30 may acquire the first evaluation index X1 for differentiating the first causative disease and acquire the second evaluation index X2 for differentiating the first causative disease, and in step 53, the estimation module 30 may estimate the disease state of the first causative disease using the first evaluation function.

In step 53, the estimation module 30 may perform a process for estimating the first causative disease when the evaluation value by the evaluation function exceeds a first threshold value. Examples of the evaluation function are a process 55 for performing odds determination as described above and a process 56 for performing probability determination by using a logistic regression model as described above. The process 55 for performing odds determination will be further described below.

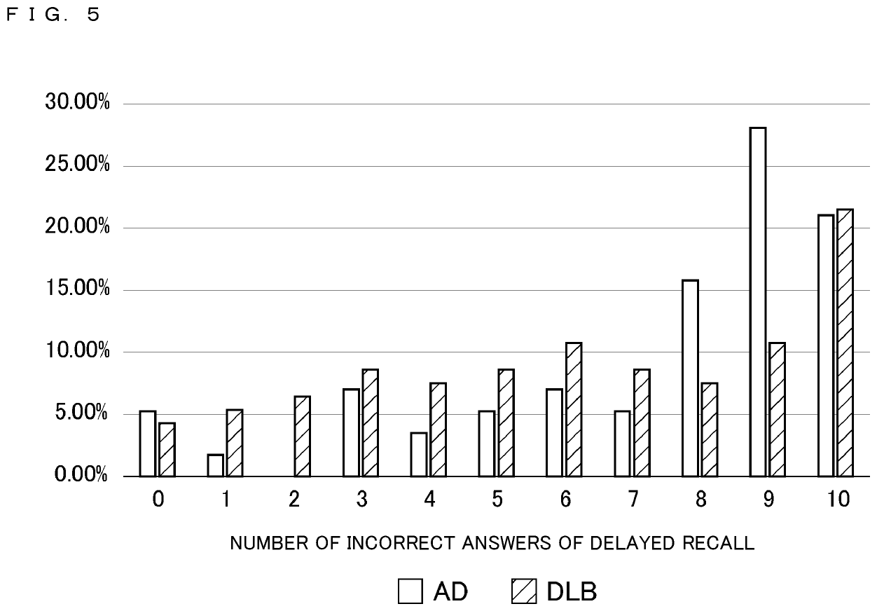

FIG. 5 shows an example of the distribution (the number of incorrect answers) of the delayed recall score of the cognitive test (ADAS-Jcog) for each disease group acquired by the second input module 20. Specifically, FIG. 5 shows the number of incorrect answers in delayed recall of subjects with AD as a causative disease and subjects with DLB as a causative disease.

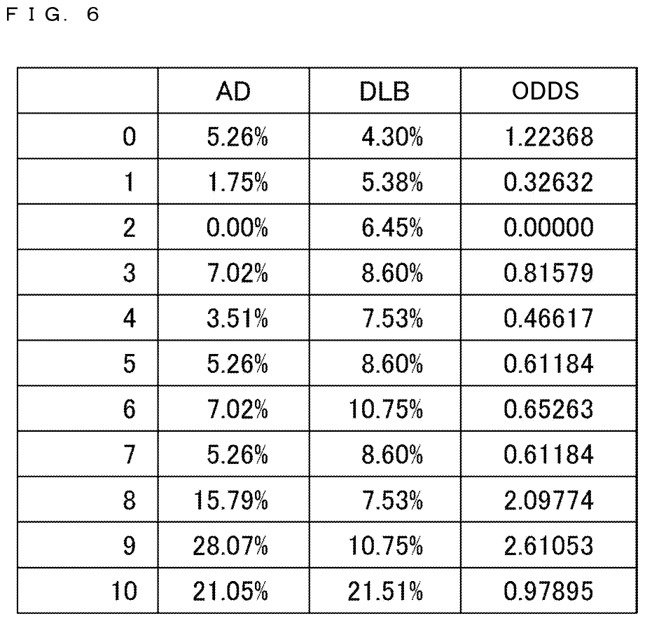

FIG. 6 shows an odds table created from the distribution of the delayed recall score of the cognitive test (ADAS-Jcog) for each disease group. At the same time, the first input module 10 acquires the output value of the activation function (softmax function) of the deep learning differentiation model using brain images of each subject as its input. In the estimation module 30, the softmax value acquired from the first input module 10 is used as the first evaluation index X1 and the disease odds score obtained by inputting the clinical information (cognition test described above) result score of the same subject (patient) is used as the second evaluation index X2, and an evaluation value S indicating that the causative disease is AD is obtained by performing multiplication of the indices X1 and X2 by the first evaluation function f1a.

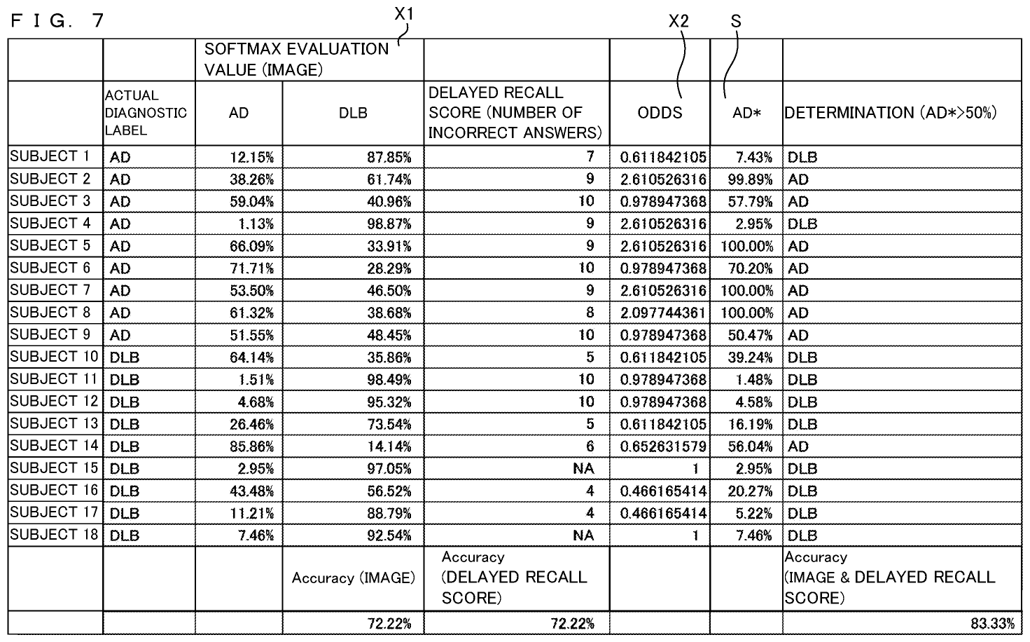

FIG. 7 shows the first evaluation index X1, the second evaluation index X2, and the evaluation value S obtained as described above. When the evaluation value S to be evaluated is equal to or greater than a cutoff value (threshold value, 50% in this example), it is possible to determine whether or not there is a brain disease, AD in this example. In this example, as a result, the determination accuracy that was 72% for the image alone increased to 83% by diagnosing the disease by using the first evaluation index X1 and the second evaluation index X2 together with each other.

In addition, there is a method in which the odds table is constructed by taking the ratio between the percentage of total of the cognitive scores of subjects in the disease group used as learning data and that in the same control group. In this example, for the determination of Alzheimer's disease and dementia with Lewy bodies, MRI images and the ADAS-Jcog delayed recall score are used for evaluation, but the present invention is not limited to this.

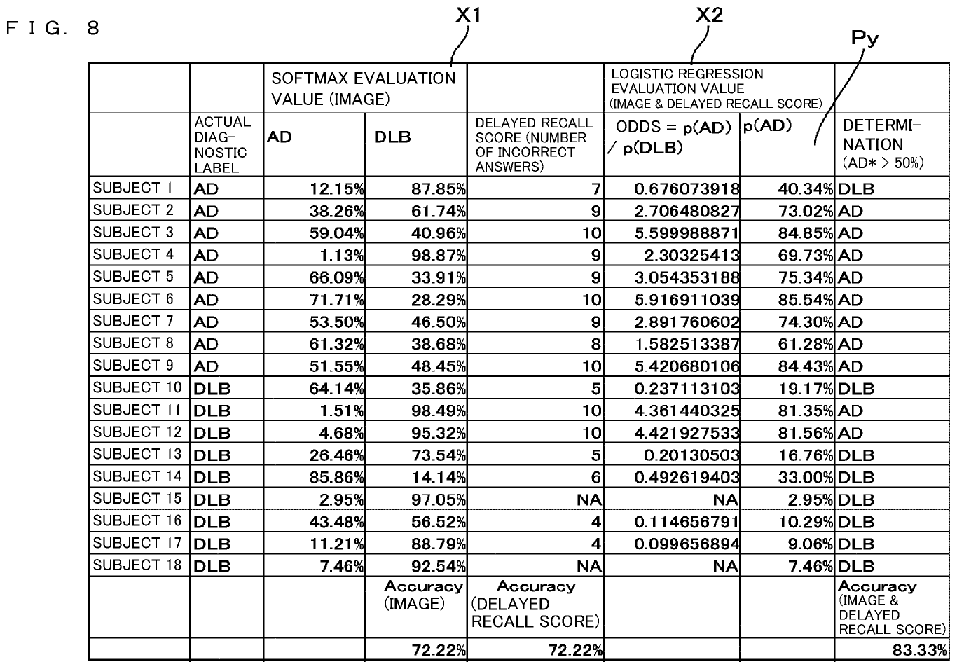

FIG. 8 shows the first evaluation index X1, the second evaluation index X2, and an evaluation value py obtained by logistic regression. When the evaluation value py to be evaluated is equal to or greater than a cutoff value (threshold value, 50% in this example), it is possible to determine whether or not there is a brain disease, AD in this example. In this example, as a result, the determination accuracy that was 72% for the image alone increased to 83% by diagnosing the disease by using the first evaluation index X1 and the second evaluation index X2 together with each other.

In addition, in the input of the logistic regression model, for the determination of Alzheimer's disease and dementia with Lewy bodies, MRI images and the ADAS-Jcog delayed recall score are used for evaluation, but the present invention is not limited to this.

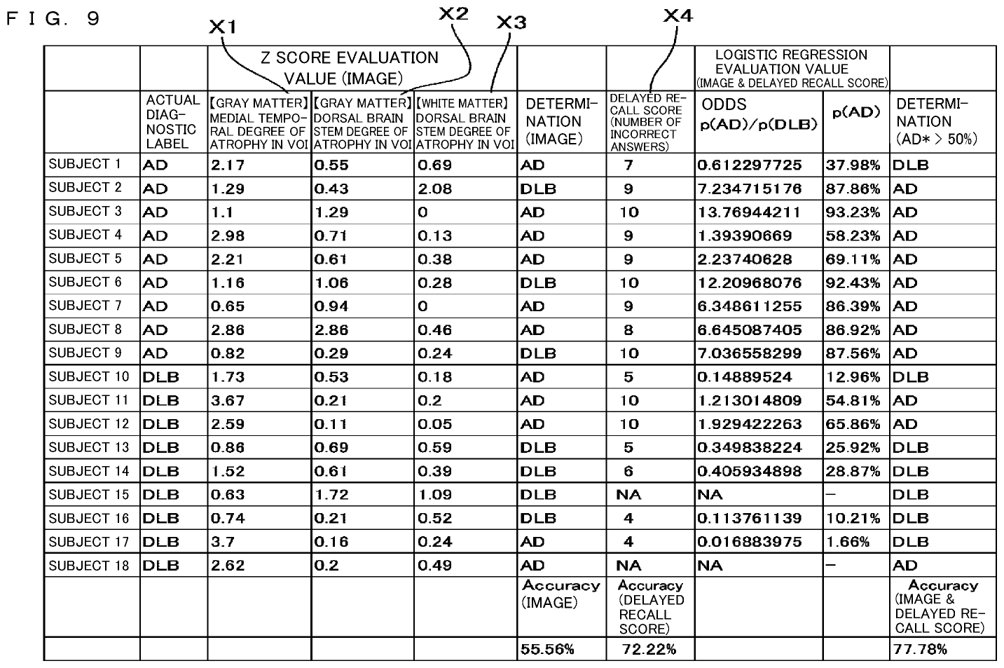

FIG. 9 shows the evaluation value py evaluated by inputting a value, which is obtained by calculating the degree of atrophy for each of the three different brain parts as the first evaluation indices X1, X2, and X3 using the Z-score, to the logistic regression together with the second evaluation index X4. When the evaluation value py to be evaluated is equal to or greater than a cutoff value (threshold value, 50% in this example), it is possible to determine whether or not there is a brain disease, AD in this example. In this example, as a result, the determination accuracy that was 56% for the image alone increased to 78% by diagnosing the disease by using the first evaluation indices X1 to X3 and the second evaluation index X4 together with each other.

In addition, in the input of the logistic regression model, for the determination of Alzheimer's disease and dementia with Lewy bodies, the Z-score and the ADAS-Jcog delayed recall score are used for evaluation, but the present invention is not limited to this.

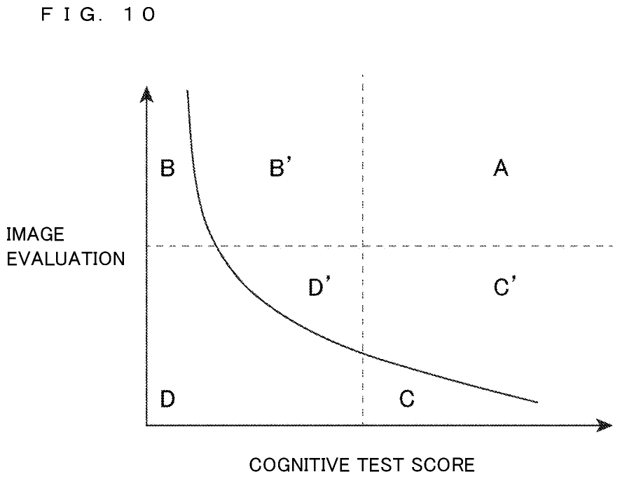

FIG. 10 shows a case of determining Negative/Positive of each of the image evaluation and the cognitive test score for a certain disease or its degree. For a certain disease or its degree, the following four categories are obtained by determining Negative/Positive of each of the image evaluation and the cognitive test score.

Category A: image (Positive) and cognitive test (Positive)

Category B: image (Negative) and cognitive test (Positive)

Category C: images (Positive) and cognitive test (Negative)

Category D: image (Negative) and cognitive test (Negative)

There is a method of determining the disease using a cutoff as an evaluation index for brain images. For example, a method is known in which the degree of hypoperfusion (CIScore) at a specific part of the occipital lobe is evaluated for patients with Alzheimer's disease and subjects with dementia with Lewy bodies and the cutoff value is used for differentiation. A method of determining an attribute according to the magnitude of the evaluation index of a brain image, in addition to setting the cutoff or separately from the cutoff, may be provided. As for the evaluation of the brain disease using the cognitive test score, it is known that a healthy person, a mild dementia patient, and an Alzheimer's disease patient can be differentiated, for example, by CDR or MMSE cutoff values. By using the evaluation values obtained by the dementia evaluation system 1 and the evaluation method described above, it is possible to subdivide the categories of D and D′ in FIG. 10 (category concept) with higher accuracy. In addition, by using the type or progression of brain disease evaluated by integrating both the evaluation of brain image alone and clinical information (for example, a cognitive test) result score, subjects are categorized and subjects for evaluation of drug efficacy are determined, and these can be used as information for performing evaluation analysis for each category.

The system 1 may include a module for outputting the estimation (evaluation) of the estimation module 30 described above and/or the background to the estimation, the first evaluation index X1, the second evaluation index X2, and other pieces of information xi to output media including a smartphone, a PC, a tablet, and paper. The output form may be characters, graphics, or images, or may be information encrypted into a QR code (registered trademark) or the like or information indicating the information access destination.

In addition, the system 1 may include a module for classifying subjects into predetermined categories based on the estimation by the estimation module 30 and/or the background to the estimation, the first evaluation index X1, the second evaluation index X2, and other pieces of information xi. In drug discovery research and stratified medicine, it is known to classify patients belonging to a certain disease into several subgroups using biomarkers and perform treatment or evaluation suitable for each subgroup. The estimation ratings of the estimation module 30 are also available as stratification markers.

### Second Embodiment

In a second embodiment, the equation (f1b) to convert the logid {log(p/(1−p))} into a probability using a sigmoid function is used as a first evaluation function. That is, the affection probability p output from the logistic regression model is used as an evaluation value. In the second embodiment, a configuration using ridge regression instead of logistic regression will be described. In the ridge regression, the evaluation value that is output is not a probability but a scalar.

Assuming that at least one of the first evaluation index X1 and the second evaluation index X2 is an explanatory variable xi and the weighting factor of each explanatory variable xi is wi, the estimation module 30 can calculate an estimated value y (hat) output as an evaluation value based on Equation (1) as the first evaluation function. In addition, x0=1, and w0 is an intercept.

$$\begin{matrix}[ Equation 6 ] & \\ \begin{matrix}\hat{y} = {w}_{0} {x}_{0} + {w}_{1} {x}_{1} + ⋯ + {w}_{n} {x}_{n} \\ = {{\sum }_{i = 0}}^{N} {w}_{i} {x}_{i}\end{matrix} & ( 1 )\end{matrix}$$

Disease labels to be separated from each other are assumed to be −1 and 1. For example, it is assumed that the label −1 indicates a healthy person and the label 1 indicates dementia. Unknown data xi can be classified according to whether y (hat) is greater than 0 or smaller than 0.

The weighting factor wi in Equation (1) can be calculated by solving an optimization problem that minimizes the loss function F shown in Equation (2). In Equation (2), k=1, n is the number of data samples, and yk is a measured value. β is a parameter that can be set in advance, and determines the magnitude of the influence of the regularization term expressed as the square of the L2 norm of wi.

$$\begin{matrix}[ Equation 7 ] & \\ F = \frac{1}{n} {\sum }_{k = 1}^{n} {( {y}_{k} - \hat{{y}_{k}} )}^{2} + β {\sum }_{i = 0}^{N} {( {w}_{i} )}^{2} & ( 2 )\end{matrix}$$

As described above, the estimation module 30 can estimate the state of the subject's brain disorder including dementia based on the evaluation value obtained by the first evaluation function having the first evaluation index and the second evaluation index as its explanatory variables. Here, the first evaluation function is expressed by a linear combination of explanatory variables with weighting factors corresponding to the respective explanatory variables as coefficients, which is predicted by ridge regression using learning data, as shown in Equation (1). In addition, the explanatory variable may be only one of the first evaluation index and the second evaluation index.

By using ridge regression, it is possible to solve the problem of multicollinearity in which the calculation of estimated values becomes unstable when there are many explanatory variables, for example, when explanatory variables are correlated with each other.

### Third Embodiment

In a third embodiment, in order to evaluate the risk of degenerative brain disease, a system for evaluating each part (region of interest) of the brain of the subject's brain image (MR, SPECT, PET, and the like) and a system for evaluating the brain disease risk for the entire brain based on the evaluation value of the subject's characteristic part will be described. In addition, the MR image is also called an MRI image. MRI images include, for example, a T1-weighted image, a T2-weighted image, a diffusion-weighted image, a FLAIR image, a diffusion tensor image, a QSM image, a pseudo-PET image, and a pseudo-SPECT image.

Brain diseases include dementia (including AD, DLB, frontotemporal lobar degeneration (FTLD), normal pressure hydrocephalus (NPH), and the like), brain tumor, mental disorders (also referred to mental illnesses; including schizophrenia, epilepsy, mood disorders, dependent personality disorder, higher brain dysfunction, and the like), Parkinson's disease, Asperger's syndrome, attention-deficit/hyperactivity disorder (ADHD), sleep disorders, childhood diseases, ischemic brain disorders, mood disorders (including depression and the like), and the like. In addition, brain disorders include dementia, multiple sclerosis, and the like as diseases relevant to the brain and includes, as diseases relevant to amyloid β, for example, neurodegenerative diseases such as mild cognitive impairment (MCI), mild cognitive impairment due to Alzheimer's disease (MCI due to AD), prodromal AD, pre-onset stage of Alzheimer's disease/preclinical AD, Parkinson's disease, multiple sclerosis, insomnia, sleep disorders, cognitive decline, cognitive dysfunction, and amyloid positive/negative diseases.

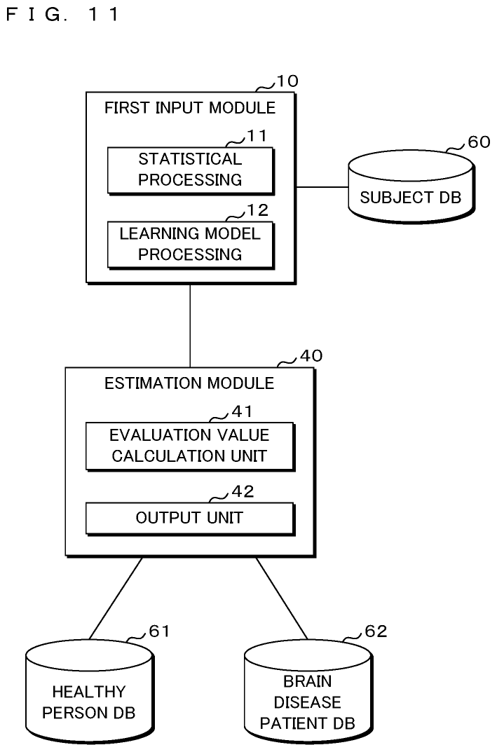

FIG. 11 is a schematic diagram showing a first example of the configuration of a system according to the third embodiment. The system (also referred to as an “evaluation system” or a “dementia evaluation system”) includes a first input module 10 and an estimation module 40. The first input module 10 has the same configuration as in the case of the first embodiment. The estimation module 40 includes an evaluation value calculation unit 41 and an output unit 42. The first input module 10 can access necessary information by referring to a subject DB 60. The subject DB 60 may be the database 19 of the first embodiment. The estimation module 40 can access necessary information by referring to a healthy person DB 61 and a brain disease patient DB 62.

The first input module 10 includes a first evaluation unit 11 and a second evaluation unit 12. The first evaluation unit 11 calculates a first evaluation index X1 by statistically evaluating the medical image of each part (region of interest) of the subject's brain. The second evaluation unit 12 outputs the first evaluation index X1 for each part (region of interest) of the subject's brain based on the medical image by using a learned model that is machine-learned so as to output the first evaluation index X1 for each part (region of interest) of the subject's brain. In addition, only one of the first evaluation unit 11 and the second evaluation unit 12 may be used, or both the first evaluation unit 11 and the second evaluation unit 12 may be used. The first input module 10 outputs the first evaluation index X1 for each part (region of interest) of the subject's brain to the estimation module 40. Hereinafter, the Z-score value of the gray matter volume value of the region of interest on the anatomical standard space will be described as the first evaluation index X1. However, the first evaluation index X1 is not limited to the Z-score.

The Z-score of a specific part of the brain can be calculated by the following equation for each pixel of the part.

Z-score=(pixel value of part of subject's brain−mean value of part of healthy person)/(standard deviation of part of healthy person)

The Z-score of the part can be calculated as a mean value of the positive Z-scores for each pixel of the part. The Z-score indicates the extent (degree) to which the pixel value of a part of a subject deviates from the pixel value of a part of the brain of a healthy person. In the case of an MR image, a higher Z-score value indicates more atrophy compared with healthy persons. Examples of a part (region of interest) of the brain include diencephalon, superior parietal lobule, inferior parietal lobule, globus pallidus, cerebellum, paracentral lobule, hippocampus, parahippocampal gyrus, precuneus, lateral ventricle, amygdala, entorhinal cortex, and brainstem, but the part (region of interest) of the brain is not limited thereto.

The first input module 10 can calculate Extent and Ratio for each part. Extent indicates the ratio of the number of voxels with a Z-score of 2 or more within a part to the total number of voxels within the part. When the Z-score is 2 or more, the Z-score is at least twice the standard deviation from the mean value of the pixel values, so that a statistically significant difference is recognized. Ratio is a value obtained by dividing the mean Z-score within a part by the whole-brain mean Z-score. The first input module 10 outputs the calculated Extent and Ratio to the estimation module 40.

The output unit 42 has a function as an output unit, and outputs display data to be displayed on a display device (not shown). The display device may be built into the system or may be a device outside the system.

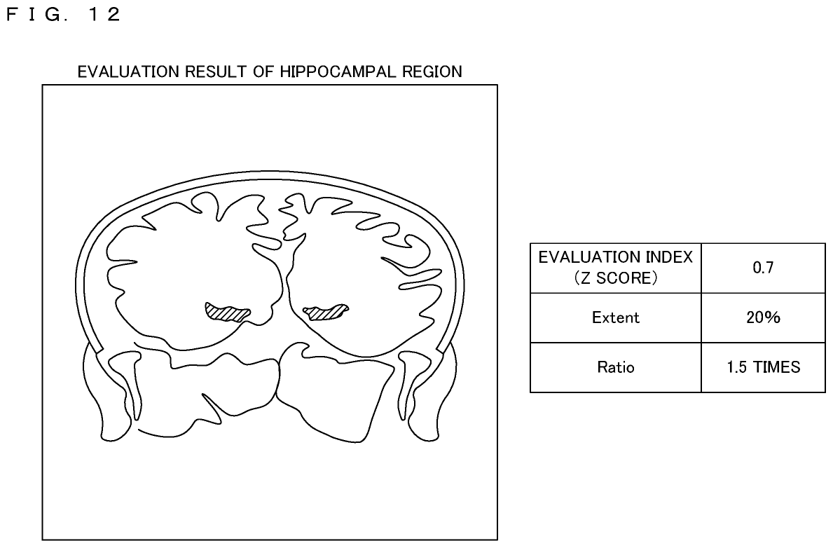

FIG. 12 is a schematic diagram showing an example of the evaluation result of a hippocampal region. FIG. 12 schematically shows an image of the subject's brain viewed from the front, and this may be different from the actual image. In the diagram, patterned parts are the left and right hippocampi, and are displayed in a map form (evaluation index map) by changing the color, pattern, and the like according to the value of the first evaluation index (Z-score). In the example of FIG. 12, the Z-score is 0.7, Extent is 20%, and Ratio is 1.5 times. In addition, the Z-score is calculated by treating each of the left and right hippocampal regions as one region. In addition, the numerical values shown in FIG. 12 are numerical values for convenience, and may be different from actual values.

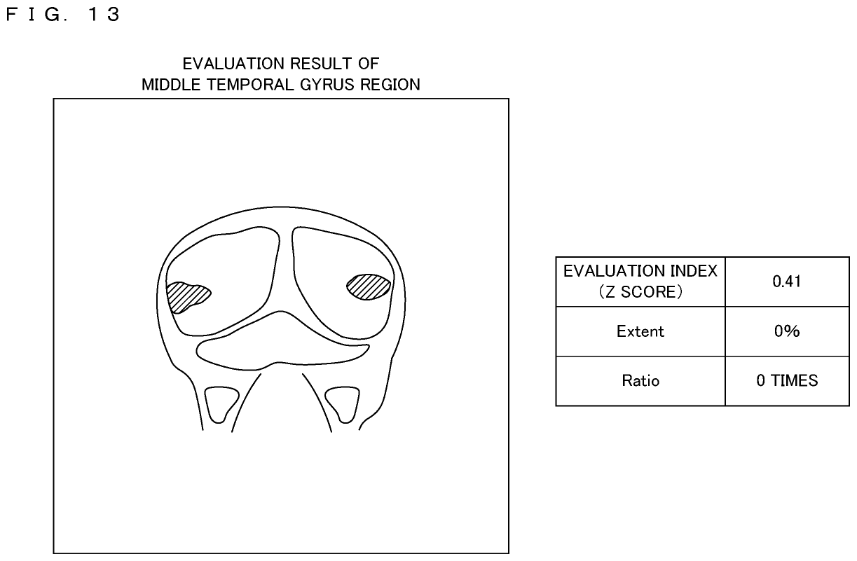

FIG. 13 is a schematic diagram showing an example of the evaluation result of a middle temporal gyrus region. FIG. 13 schematically shows a cross-sectional image of the middle temporal gyms of the subject, and may be different the actual image. In the diagram, patterned parts are the left and right middle temporal gyri, and are displayed in a map form by changing the color, pattern, and the like according to the value of the first evaluation index (Z-score). In the example of FIG. 13, the Z-score is 0.41, Extent is 0%, and Ratio is 0 times. In addition, the Z-score is calculated by treating each of the left and right middle temporal gyms regions as one region. In addition, the numerical values shown in FIG. 13 are numerical values for convenience, and may be different from the actual ones.

Parts other than the hippocampal region and the middle temporal gyms region can be similarly displayed. By providing a doctor or the like with the evaluation results shown in FIGS. 12 and 13, the doctor or the like can use the evaluation results as materials for diagnosing various brain diseases, including dementia, of subjects.

The evaluation value calculation unit 41 has a function as a calculation unit, and calculates a region evaluation value (also referred to as a “part evaluation value”) for each of a plurality of regions of interest based on a first evaluation index for each of a plurality of parts (regions of interest) of the subject's brain and a weighting factor corresponding to each first evaluation index.

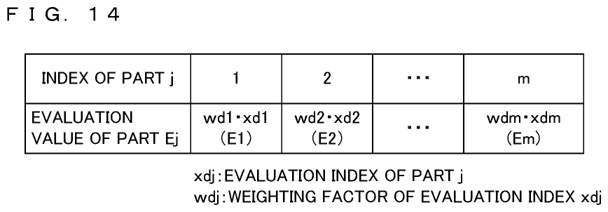

FIG. 14 is a schematic diagram showing an example of the evaluation value of each part. As shown in FIG. 14, it is assumed that the index of a part is j and the number of parts is m. Each part can be expressed by j=1, 2, . . . , m. It is assumed that the evaluation index of each part j is xdj and the weighting factor of the evaluation index xdj is wdj. The evaluation value Ej of each part can be expressed by E1=wd1·xd1, E2=wd2·xd2, Em=wdm·xdm.

The output unit 42 can output the evaluation value of the part calculated by the evaluation value calculation unit 41. The output unit 42 may output display data for displaying the evaluation values of a plurality of parts of the subject's brain so as to be arranged in a predetermined order (for example, in descending order of evaluation values). When receiving the selection of a required subject from a plurality of subjects, the evaluation value calculation unit 41 can calculate the evaluation value of each of a plurality of parts of the brain of the selected subject.

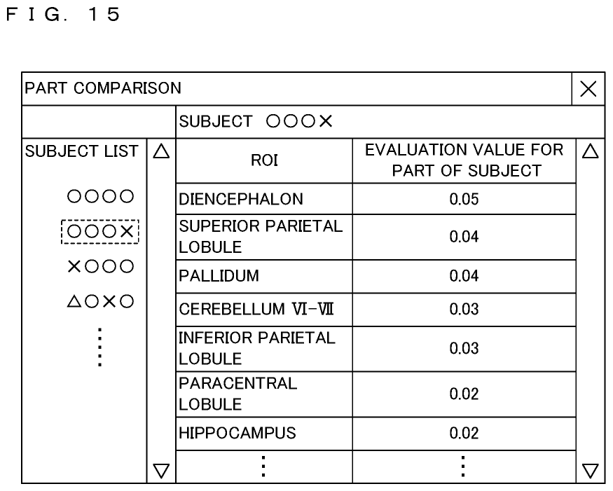

FIG. 15 is a schematic diagram showing a first example of a part comparison screen. The part comparison screen has an area for displaying a list of subjects and an area for displaying an evaluation value for each part (ROI) of the subject. In the list of subjects, the IDs, names, and the like of a plurality of subjects are displayed in a list, so that it is possible to select a target subject. For example, a doctor or the like can select a subject to be diagnosed from the list. In the example of FIG. 15, a subject OOOX surrounded by a dashed line is selected. The evaluation values of the parts of the subjects can be displayed so as to be arranged in descending order of evaluation values. In the example of FIG. 15, the evaluation value of the diencephalon is 0.5, which is the largest value, and is therefore displayed at the top. Therebelow, the ROI and the evaluation value of each part are displayed in descending order of evaluation values so as to be associated with each other. Therefore, when the subject suffers from a specific brain disease, it is possible to determine which part of the brain is the cause of the brain disease. In addition, the numerical values illustrated in FIG. 15 are values for convenience, and may be different from actual values.

By appropriately selecting a subject on the screen shown in FIG. 15, it is possible to dynamically switch between a plurality of subjects and display the evaluation values of the parts.

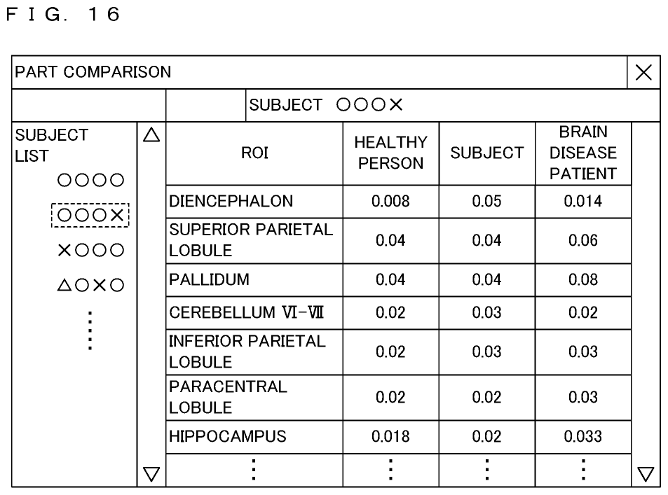

FIG. 16 is a schematic diagram showing a second example of the part comparison screen. The difference from the first example illustrated in FIG. 15 is that the evaluation value of each part of a healthy person and the evaluation value of each part of a specific brain disease patient are displayed. The estimation module 40 has a function as a healthy person evaluation value acquisition unit, and can acquire an evaluation value for each of a plurality of parts of a healthy person (for example, an average of evaluation values of a large number of healthy people) from the healthy person DB 61. In addition, the estimation module 40 has a function as a brain disease patient evaluation value acquisition unit, and can acquire an evaluation value for each of a plurality of parts of a brain disease patient suffering from a specific brain disease (for example, an average of evaluation values of a large number of brain disease patients suffering from a specific brain disease) from the brain disease patient DB 62. The estimation module 40 can selectively acquire an evaluation value for each of a plurality of parts of a patient with a specific brain disease among a plurality of types of brain disease.

In FIG. 16, the evaluation value of each part of both a healthy person and a brain disease patient is displayed, but the evaluation value of each part of either a healthy person or a brain disease patient may be displayed. That is, the output unit 42 can output display data for displaying the evaluation values of a plurality of parts of a subject and a healthy person in a display mode that allows comparison. In addition, the output unit 42 can output display data for displaying the evaluation values of a plurality of parts of a subject and a brain disease patient in a display form that allows comparison. In addition, although not shown, a list of a plurality of types of brain diseases may be displayed on the part comparison screen shown in FIG. 16 so that a required brain disease can be selected from the brain diseases displayed in the list, and the evaluation value of each part of the brain disease patient suffering from the selected brain disease may be displayed each time a brain disease is selected.

As shown in FIG. 16, by displaying the evaluation value of each part of the subject and the evaluation value of each part of at least one of the healthy person and the brain disease patient in a display mode that allows comparison, it is possible to determine the brain disease state of the subject in comparison with the healthy person or the brain disease patient.

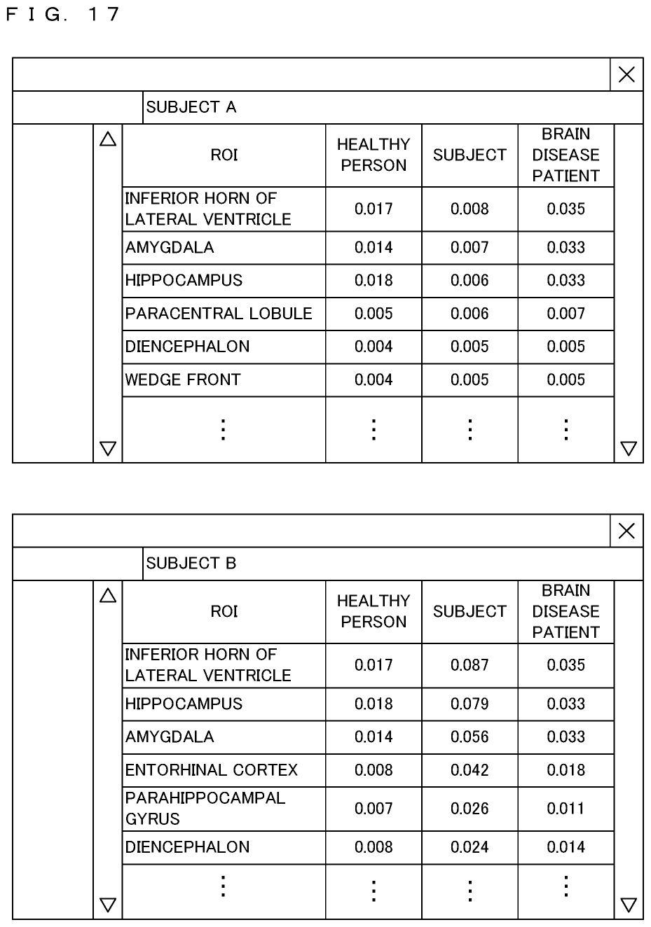

FIG. 17 is a schematic diagram showing a third example of the part comparison screen. The example of FIG. 17 shows that it is possible to estimate a brain disease of a subject based on the evaluation value for each part of the subject. In subjects with core symptoms of dementia, there is a suggestive feature of image findings showing “relatively mild medial temporal atrophy in dementia with Lewy bodies (DLB) compared with Alzheimer's dementia (AD)”. Based on the evaluation value relevant to atrophy for each part of the subject, it is possible to determine the above-described suggestive feature. Here, the medial temporal region is a gray matter region including the parahippocampal gyms and the hippocampus. As shown in FIG. 17, in the case of subject A, the evaluation of hippocampal atrophy appears at the top, but the evaluation value of the subject A is smaller than the evaluation value of the brain disease patient. Therefore, it can be determined that the atrophy state is mild. On the other hand, in the case of subject B, the evaluation values for the hippocampus and the parahippocampal gyrus are large. Therefore, it can be determined that atrophy is progressing. In such a case, the subject A can be presumed to have dementia with Lewy bodies, and subject B can be presumed to have Alzheimer's dementia.

Next, the evaluation of the disease risk for the whole brain of a subject based on the evaluation value for each part of the brain of the subject will be described.

The estimation module 40 has a function as an estimation unit, and estimates the state of the subject's brain disorder including dementia based on the whole-brain evaluation value obtained by the second evaluation function whose variable is the first evaluation index of each of a plurality of parts of the subject's brain. Specifically, the evaluation value calculation unit 41 can calculate the whole-brain evaluation value by using the second evaluation function.

The second evaluation function can be expressed by Equation (3).

$$\begin{matrix}[ Equation 8 ] & \\ \begin{matrix}EA = {wd}_{1} \cdot x {d}_{1} + {wd}_{2} \cdot {xd}_{2} \cdot {xd}_{2} + ⋯ + {wd}_{m} \cdot {xd}_{m} + ε \\ = {{\sum }_{j = 1}}^{m} {wd}_{j} \cdot {xd}_{j} + ε\end{matrix} & ( 3 )\end{matrix}$$

$$\begin{matrix}[ Equation 9 ] & \\ L = {{\sum }_{i = 1}}^{n} {( E - EA )}^{2} + α {{\sum }_{j = 1}}^{m} {( {wd}_{j} )}^{2} & ( 4 )\end{matrix}$$

In Equation (3), EA is a predicted value of the whole-brain evaluation value, xdj is an evaluation index of a part j, and wdj is a weighting factor of the evaluation coefficient xdj. m is the number of parts, and ε is a constant for evaluating the error.

The weighting factor wdj in Equation (3) can be calculated by solving an optimization problem that minimizes the loss function L shown in Equation (4). In Equation (4), i=1, . . . , n is the number of learning data samples, and E is a measured value of the whole-brain evaluation value. α is a parameter that can be set in advance, and determines the magnitude of the influence of the regularization term expressed as the square of the L2 norm of wdj. That is, the second evaluation function expressed by Equation (3) is expressed by a linear combination of the first evaluation index xdj with the weighting factor wdj corresponding to each first evaluation index xdj as a coefficient, which is predicted by ridge regression using learning data.

The labels of classes to be separated from each other are assumed to be −1 and 1. For example, it is assumed that the label −1 indicates a healthy person and the label 1 indicates dementia. The unknown evaluation index xdj can be classified according to whether the whole-brain evaluation value EA is greater than 0 or smaller than 0. In addition, if there are three or more classes to be separated from each other (for example, the three classes are assumed to be a healthy person, brain disease B1, and brain disease B2), brain diseases may be classified by majority vote for all possible combinations of two classes. Specifically, assuming that the brain disease B1 is classified twice, the brain disease B2 is classified once, and the healthy person is classified 0 times by three combinations of the healthy person and the brain disease B1, the brain disease B1 and the brain disease B2, and the brain disease B2 and the healthy person, the unknown evaluation index xdj can be classified into the brain disease B1 having the highest frequency.

As described above, it is possible to solve the problem of multicollinearity by using the ridge regression model. Hereinafter, explanation on this point will be given.

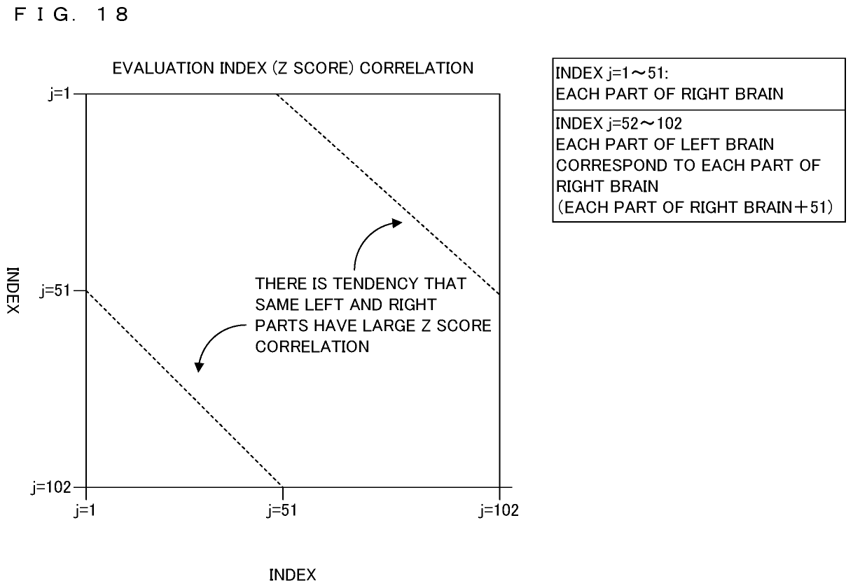

FIG. 18 is a schematic diagram showing an example of Z-score correlation between each part of the left brain and each part of the right brain. j is an index indicating a part. Each part of the right brain is expressed by j=1 to 51. Each part of the left brain is expressed by j=52 to 102. Here, the part j of the right brain corresponds to the part (j+51) of the left brain. For example, if the part j of the right brain is the hippocampus, the part (j+51) of the left brain is also the hippocampus. In FIG. 18, the dashed straight line indicates a part with a high Z-score correlation. That is, there is a tendency that the same left and right parts have a large Z-score correlation.

If a function based on the logistic regression model is used as the second evaluation function, the variables in the logistic regression model are correlated with other variables, causing the problem of multicollinearity. For this reason, it becomes unstable to calculate the estimated value. Therefore, by using the ridge regression model expressed by Equation (3), a regularization term is added, so that it is possible to solve the problem of multicollinearity. Specifically, when a function based on the logistic regression model is used, a large number of parts appear in which one of the right brain and the left brain has a positive weighting factor and the other has a negative weighting factor in the same part of the right brain and the left brain. For this reason, it is impossible to accurately calculate the whole-brain evaluation value. Essentially, all weighting factors are expected to be positive factors. By using the function based on the ridge regression model, it is possible to greatly reduce the number of parts with negative weighting factors. For example, it is possible to calculate the whole-brain evaluation value with an accuracy of about 82%.

Next, a method for further improving the estimation accuracy of the whole-brain evaluation value when using the ridge regression model will be described.

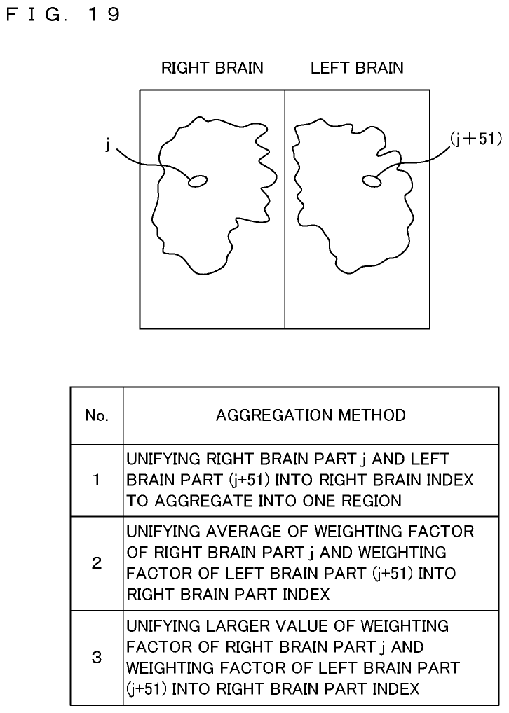

FIG. 19 is a schematic diagram showing an example of a left-brain and right-brain aggregation method. As shown in FIG. 19, the part of the right brain is expressed by j, and the same part of the left brain as the right brain is expressed by (j+51). The first aggregation method is a method of unifying the right brain part j and the left brain part (j+51) into a right brain index j to aggregate these into one region. Assuming that each of the right brain and the left brain has 51 parts and the total number of parts in the brain is 102, the number of parts is reduced from 102 to 51 by the first aggregation method. By ridge regression using the first aggregation method, the accuracy of estimating the whole-brain evaluation value could reach about 96%. In addition, the number of parts in the whole brain is not limited to 102, and other numerical values may be used.

The second aggregation method is a method of unifying the average of the weighting factor of the right brain part j and the weighting factor of the left brain part (j+51) into the right brain part index. Assuming that each of the right brain and the left brain has 51 parts and the total number of parts in the brain is 102, the number of parts is reduced from 102 to 51 by the second aggregation method. By ridge regression using the second aggregation method, the accuracy of estimating the whole-brain evaluation value could reach about 91%.

The third aggregation method is a method of unifying the larger value of the weighting factor of the right brain part j and the weighting factor of the left brain part (j+51) into the right brain part index. Assuming that each of the right brain and the left brain has 51 parts and the total number of parts in the brain is 102, the number of parts is reduced from 102 to 51 by the third aggregation method. By ridge regression using the third aggregation method, the accuracy of estimating the whole-brain evaluation value could reach about 82%.

Next, a specific example of the whole-brain evaluation value EA calculated by the evaluation value calculation unit 41 will be described.

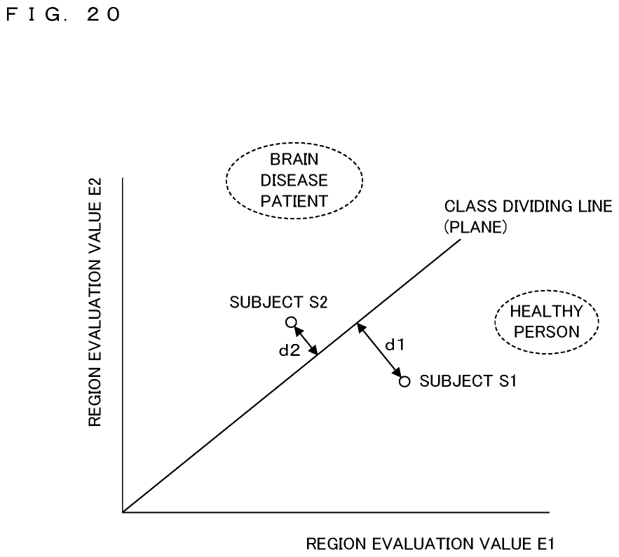

FIG. 20 is a schematic diagram showing a specific example of the whole-brain evaluation value. As described above, the whole-brain evaluation value EA can be calculated by Equation (3). When the above-described aggregation method is used, the number j of parts can be set to, for example, 51, and the number of evaluation values for the parts is also 51. In FIG. 20, for the sake of simplification, the meaning of the whole-brain evaluation value will be explained on a two-dimensional plane by setting the number of parts to two. As shown in FIG. 20, the region evaluation values are E1 and E2. A distance from the class dividing line (plane) that divides the classes (healthy person, brain disease patient) on the two-dimensional plane is the whole-brain evaluation value. For example, −d1 indicates the whole-brain evaluation value in the case of subject S1, and d2 indicates the whole-brain evaluation value in the case of subject S2. Here, a negative sign is given to the healthy person, and a positive sign is given to the brain disease patient. By using the subject's whole-brain evaluation value, it is possible to visually express whether the subject is close to a healthy person or a brain disease patient, as shown below.

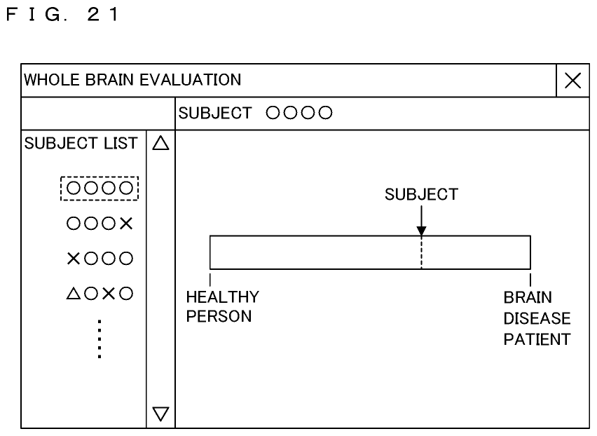

FIG. 21 is a schematic diagram showing a first example of a whole-brain evaluation screen. The whole-brain evaluation screen has an area for displaying a list of subjects and an area for displaying the whole-brain evaluation values of the subjects. In the list of subjects, the IDs, names, and the like of a plurality of subjects are displayed in a list, so that it is possible to select a target subject. For example, a doctor or the like can select a subject to be diagnosed from the list. In the example of FIG. 21, a subject OOOO surrounded by a dashed line is selected.

The estimation module 40 has a function as a whole-brain evaluation value acquisition unit, and can acquire the whole-brain evaluation value of a healthy person from the healthy person DB 61 and acquire a whole-brain evaluation value regarding a required brain disease of the brain disease patient from the brain disease patient DB 62. The whole-brain evaluation value of the subject can be displayed in a display mode that allows comparison with the whole-brain evaluation values (for example, the mean value of the whole-brain evaluation values) of healthy persons and brain disease patients. In the example of FIG. 21, one end of the horizontal bar graph indicates a healthy person and the other end indicates a brain disease patient, and the subject is expressed by the position on the horizontal bar graph. This makes it possible to visually express whether the subject is close to a healthy person or a brain disease patient.

By appropriately selecting a subject on the screen shown in FIG. 21, it is possible to dynamically switch between a plurality of subjects and display the whole-brain evaluation value. In addition, although not shown, a list of a plurality of types of brain diseases may be displayed on the whole-brain evaluation screen shown in FIG. 21 so that a required brain disease can be selected from the brain diseases displayed in the list, and a horizontal bar graph showing the brain disease patient suffering from the selected brain disease at the other end may be displayed each time a brain disease is selected.

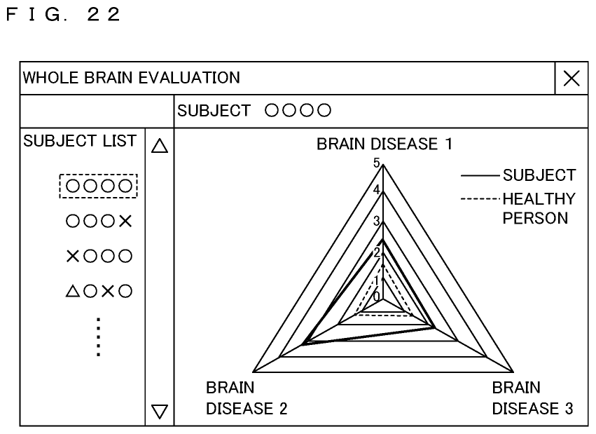

FIG. 22 is a schematic diagram showing a second example of the whole-brain evaluation screen. As shown in FIG. 22, the whole-brain evaluation value of the subject and the whole-brain evaluation values of healthy persons and brain disease patients can be displayed on a radar chart. A plurality of brain disease axes are arranged in a regular polygonal shape from the center. In the example of FIG. 22, three brain diseases, brain diseases 1, 2, and 3, are expressed by equilateral triangular radar charts. A dashed line indicates the position of a healthy person on the radar chart, and a solid line indicates the position of the subject. As a result, it is possible to visually express whether the subject is close to a healthy person or a brain disease patient for each brain disease.

Although the first example of the system illustrated in FIG. 11 is configured to use the first evaluation index X1, the second evaluation index X2 may be used in addition to the first evaluation index.

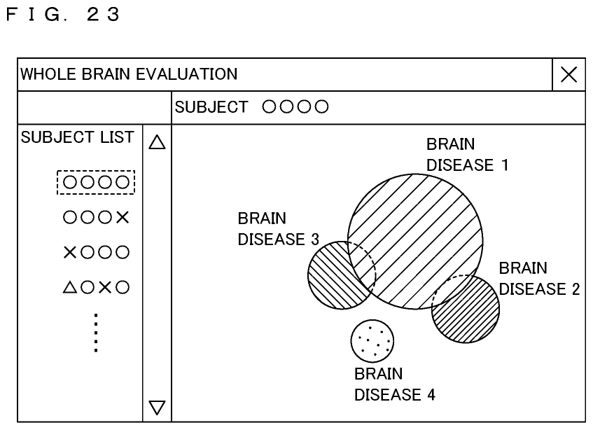

FIG. 23 is a schematic diagram showing a third example of the whole-brain evaluation screen. As shown in FIG. 23, the possibility of each brain disease (brain diseases 1, 2, 3, and 4 in the example of FIG. 23) based on the whole-brain evaluation value of the subject is visually expressed by the size of a circle like a bubble chart. For example, for each brain disease, the size of the circle decreases as the subject's whole-brain evaluation value approaches the average of the whole-brain evaluation values of healthy persons, and vice versa, the size of the circle increases as the subject's whole-brain evaluation value approaches the average of the whole-brain evaluation values of brain disease patients. In the example of FIG. 23, it can be seen that the subject OOOO is highly likely to be a brain disease patient with brain disease 1. In addition, it can be seen that the subject OOOO has a possibility of brain diseases 2 and 3 or the possibility of brain diseases 2 and 3 cannot be denied. In addition, it can be seen that the subject OOOO is, for example, at the level of a healthy person for the brain disease 4. In addition, the types of brain diseases are not limited to the four types shown in FIG. 23.

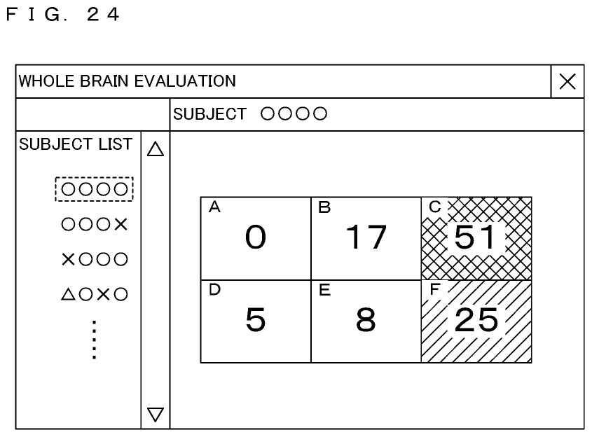

FIG. 24 is a schematic diagram showing a fourth example of the whole-brain evaluation screen. In FIG. 24, the symbols A to F of respective cells can be the types of brain diseases or the parts of the brain. The numbers in the table can indicate the strength of the relevance of each brain disease or each part. The numbers may be the subject's whole-brain evaluation values. In addition, as shown in FIG. 24, the strength of the relevance of each brain disease or each part may be expressed by the pattern or color attached to each cell. In the example of FIG. 24, it can be seen that the subject OOOO is strongly relevant to the brain disease (or part) C.

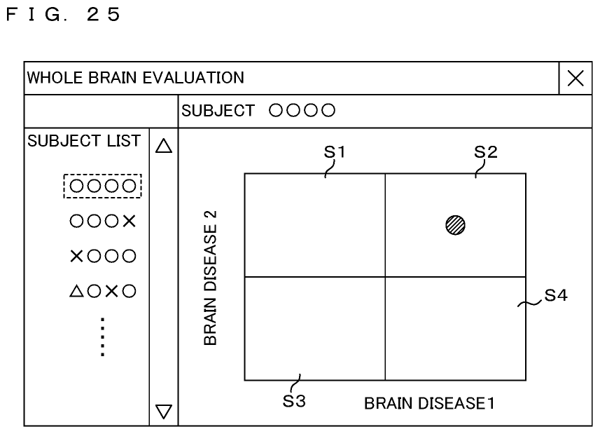

FIG. 25 is a schematic diagram showing a fifth example of the whole-brain evaluation screen. As shown in FIG. 25, the possibility of each brain disease (brain diseases 1 and 2 in the example of FIG. 23) based on the whole-brain evaluation value of the subject is visually expressed by a matrix chart. The whole-brain evaluation screen is divided into four regions S1 to S4 by a two-axis matrix. The region S1 indicates that the possibility of the brain disease 2 is high and the possibility of the brain disease 1 is low (that is, it can be determined that the subject has the brain disease 2). The region S2 indicates that the possibility of the brain disease 2 is high and the possibility of the brain disease 1 is also high (that is, it can be determined that the patient has both the brain diseases 1 and 2). The region S3 indicates that the possibility of the brain disease 2 is low and the possibility of the brain disease 1 is also low (that is, it can be determined that the patient has neither the brain disease 1 nor the brain disease 2). The region S4 indicates that the possibility of the brain disease 2 is low and the possibility of the brain disease 1 is high (that is, it can be determined that the subject has the brain disease 1). In the example of FIG. 25, it can be determined that the subject OOOO has both the brain diseases 1 and 2.

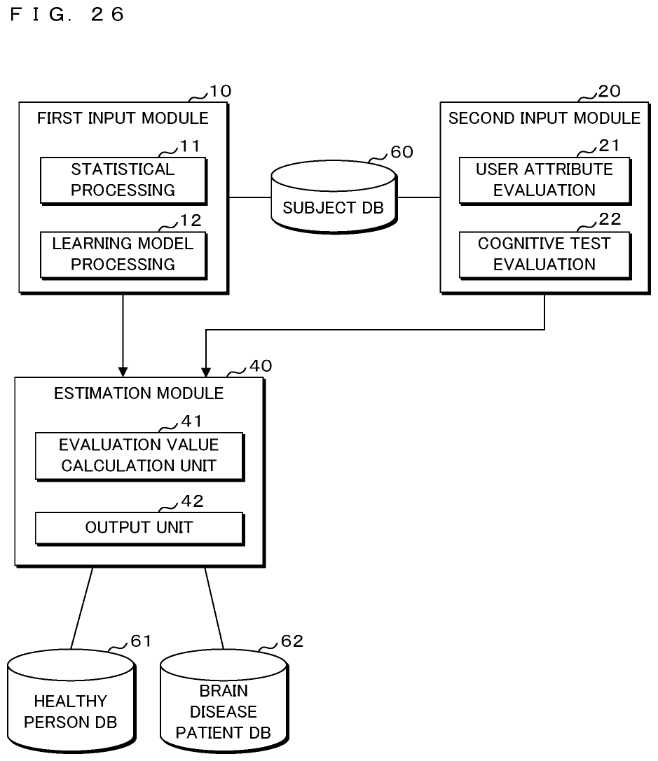

FIG. 26 is a schematic diagram showing a second example of the configuration of the system according to the third embodiment. The second example is different from the first example in that the second input module 20 is provided. Since the second input module 20 is the same as in the case of the first embodiment shown in FIG. 1, the description thereof will be omitted. The estimation module 40 can estimate the state of the subject's brain disorder including dementia based on the whole-brain evaluation value obtained by the second evaluation function whose variables are the first evaluation index and the second evaluation index X2 of each of a plurality of parts of the subject's brain. Specifically, the evaluation value calculation unit 41 can calculate the whole-brain evaluation value by using the second evaluation function. The second evaluation function can be expressed by Equation (3) described above, and the evaluation index xdj includes the first evaluation index and the second evaluation index X2.

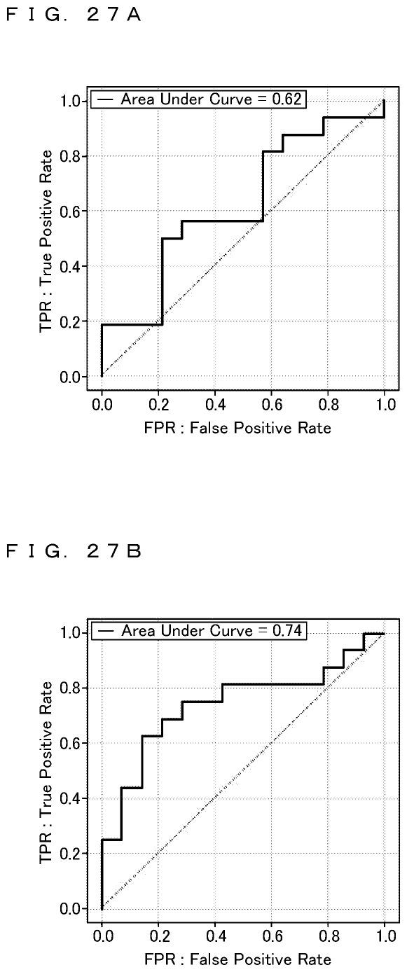

FIGS. 27A and 27B are schematic diagrams showing examples of an ROC curve. FIG. 27A shows an ROC (Receiver Operating Characteristic) curve when AD and DLB are differentiated by using only the first evaluation index (Z-score of 51 parts), and FIG. 27B shows an ROC curve when AD and DLB are differentiated by using the second evaluation index (delayed recall score in ADAS Cog test) in addition to the first evaluation index. In FIGS. 27A and 27B, the vertical axis indicates the true positive rate (TPR) and the horizontal axis indicates the FPR (false positive rate). The ROC curve is obtained by calculating and plotting the true positive rate and the false positive rate for each cutoff point for distinguishing between normal and abnormal in AD diagnosis and DLB diagnosis. The area under the plotted graph is called AUC (Area Under the Curve), and the AUC can take values from 0 to 1. The closer the AUC is to 1, the higher the determination accuracy. AUC=0.62 in FIG. 27A, and AUC=0.74 in FIG. 27B. Therefore, the AUC in FIG. 27B is higher. In the example of FIG. 27, it can be seen that the determination accuracy is improved by adding the delayed recall score for evaluating the deterioration of long-term memory (strong in AD cases).

Next, an operation of the system according to the third embodiment will be described.

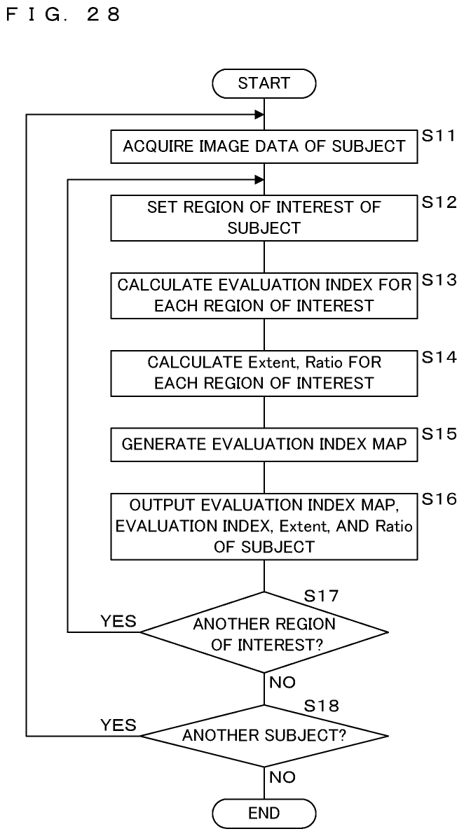

FIG. 28 is a flowchart showing the procedure of a first example of a process for outputting an evaluation result of each part of the brain. The system acquires image data of the subject's brain (S11), and sets the subject's region of interest (part) (S12). The system calculates an evaluation index (for example, Z-score) for each region of interest (S13), and calculates Extent and Ratio for each region of interest (S14).

The system generates an evaluation index map for each region of interest based on the calculated evaluation index (S15), and outputs the subject's evaluation index map, evaluation index, Extent, and Ratio (S16). The system determines whether or not there is another region of interest (S17). If there is another region of interest (YES in S17), the system continues the processing from step S12.

If there is no other region of interest (NO in S17), the system determines whether or not there is another subject (S18). If there is another subject (YES in S18), the system continues the processing from step S11, and if there is no other subject (NO in S18), the process ends.

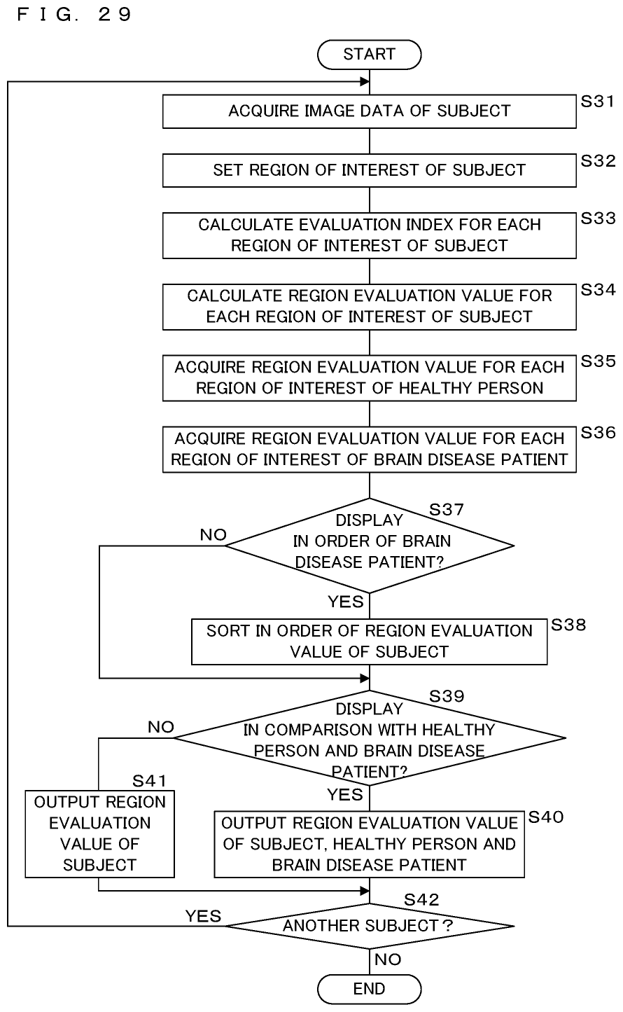

FIG. 29 is a flowchart showing the procedure of a second example of the process for outputting an evaluation result of each part of the brain. The system acquires image data of the subject's brain (S31), and sets the subject's region of interest (part) (S32). The system calculates an evaluation index (for example, Z-score) for each region of interest (S33), and calculates a region evaluation value Ej for each region of interest of the subject (S34). The region evaluation value Ej can be calculated by Ej=wdj·xdj. Here, xdj is an evaluation index (for example, Z-score) of the part j, and wdj is a weighting factor.

The system acquires a region evaluation value for each region of interest of a healthy person from the healthy person DB 61 (S35), and acquires a region evaluation value for each region of interest of a brain disease patient from the brain disease patient DB 62 (S36). The system determines whether or not to perform display in order of region evaluation values (for example, in descending order) (S37). When performing display in order of region evaluation values (YES in S37), the system performs sorting in order of subject's region evaluation values (S38), and performs the processing of step S39, which will be described later.

If the region evaluation values are not displayed in order (NO in S37), the system determines whether or not to display the subject's region evaluation values in comparison with healthy persons and brain disease patients (S39). When displaying the subject's region evaluation values in comparison with healthy persons and brain disease patients (YES in S39), the system outputs the region evaluation values of the subject, healthy persons, and brain disease patients (S40), and performs the processing of step S42, which will be described later.

If the display is not performed in comparison with healthy persons and brain disease patients (NO in S39), the system outputs the subject's region evaluation value (S41), and determines whether or not there is another subject (S42). If there is another subject (YES in S42), the system continues the processing from step S31, and if there is no other subject (NO in S42), the process ends.

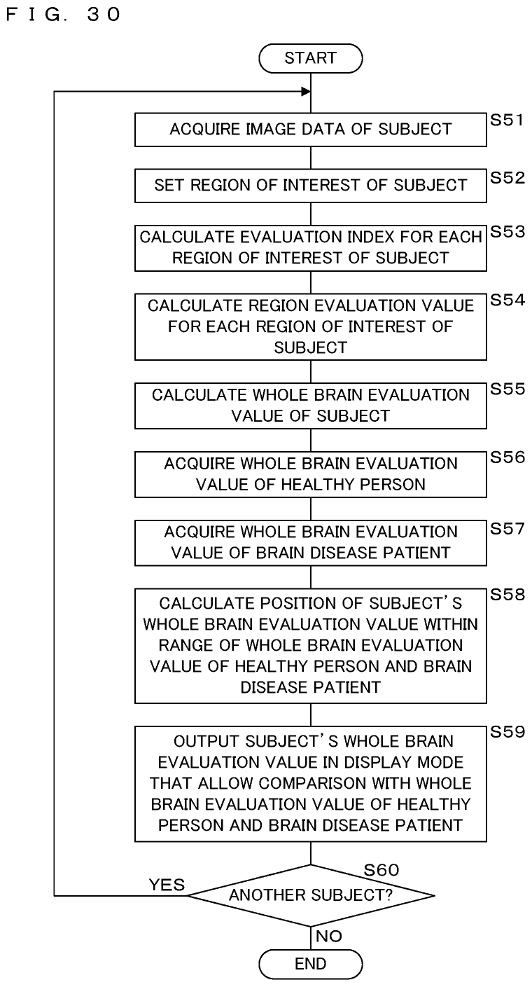

FIG. 30 is a flowchart showing the procedure of a process for outputting the whole-brain evaluation result. The system acquires image data of the subject's brain (S51), and sets the subject's region of interest (part) (S52). The system calculates an evaluation index (for example, Z-score) for each region of interest (S53), and calculates a region evaluation value Ej for each region of interest of the subject (S54). The region evaluation value Ej can be calculated by Ej=wdj·Adj. Here, xdj is an evaluation index (for example, Z-score) of the part j, and wdj is a weighting factor.

The system calculates the whole-brain evaluation value EA (S55). The whole-brain evaluation value EA can be calculated by Equation (3). The system acquires the whole-brain evaluation values of healthy persons from the healthy person DB 61 (S56), and acquires the whole-brain evaluation values of brain disease patients from the brain disease patient DB 62 (S57). The system calculates the position of the subject's whole-brain evaluation value within the range of the whole-brain evaluation values of the healthy persons and the brain disease patients (S58). For example, assuming that the average of the whole-brain evaluation values of healthy persons is a, the average of the whole-brain evaluation values of brain disease patients is b, and the whole-brain evaluation value of the subject is c, it is possible to calculate the position according to where the value c is in the range from a to b.

The system outputs the subject's whole-brain evaluation value in a display mode (see, for example, FIG. 21) that allows comparison with the whole-brain evaluation values of healthy persons and brain disease patients (S59). The system determines whether or not there is another subject (S60). If there is another subject (YES in S60), the system continues the processing from step SM, and if there is no other subject (NO in S60), the process ends.

In the third embodiment described above, the Z-score value of the gray matter volume value of the region of interest on the anatomical standard space is used as the first evaluation index X1, but the first evaluation index is not limited to the Z-score. For example, physical quantities such as a blood flow rate in the region of interest and the amount of accumulation of malignant proteins (for example, amyloid β and tau protein) in the region of interest may be used. When using such physical quantities, the percentage of physical quantities in the region of interest exceeding a predetermined threshold value (for example, the ratio of the number of voxels exceeding a threshold value to the total number of voxels in the region of interest) may be used for normalization. As a result, evaluation indices can be compared between regions of interest regardless of the size of the region of interest and the like.

In addition, at least one of SUVR and BR may be used as the first evaluation index X1. In this case, for example, at least one of the terms w_{SUVR}·SUVR and w_{BR}·BR may be added to Equation (3). Here, w_{SUVR} is the weighting factor of the evaluation index SUVR, and w_{BR} is the weighting factor of the evaluation index BR. In addition, when using the evaluation index SUVR or the evaluation index BR, if these index values are values standardized from the distribution and the like in the healthy person DB, it is possible to evaluate the degree of importance when each subject is determined to have a disease similar to the case where each part is evaluated only by the Z-score. For example, the standardized value SUVR_{Z} of SUVR can be calculated by SUVR_{Z}={(average of SUVR of healthy persons−SUVR of subject)/standard deviation of SUVR of healthy persons}.

It is to be noted that, as used herein and in the appended claims, the singular forms “a”, “an”, and “the” include plural referents unless the context clearly dictates otherwise.
As this invention may be embodied in several forms without departing from the spirit of essential characteristics thereof, the present embodiments are therefore illustrative and not restrictive, since the scope of the invention is defined by the appended claims rather than by the description preceding them, and all changes that fall within metes and bounds of the claims, or equivalence of such metes and bounds thereof are therefore intended to be embraced by the claims.

1-27. (canceled)

28. A system, comprising:
a first input module configured to acquire a first evaluation index based on data regarding a physical state of a brain of a subject;
a second input module configured to acquire a second evaluation index based on data regarding a function of the brain of the subject; and
an estimation module configured to estimate a state of a brain disorder, including dementia, of the subject based on an evaluation value obtained by a first evaluation function having the first evaluation index and the second evaluation index as its variables.

29. The system according to claim 28,
wherein the first input module includes at least one of a first evaluation unit that acquires the first evaluation index by statistically evaluating a first type medical image of a region of interest of at least a part of the subject's brain and a second evaluation unit that acquires the first evaluation index by evaluating the medical image of the subject by using a first model machine-learned to evaluate a first disease based on the first type medical image.

30. The system according to claim 28,
wherein the second input module includes a configuration for acquiring an evaluation of clinical information including a cognitive test as the second evaluation index.

31. The system according to claim 28,
wherein the subject is included in a group ingesting at least one of drugs, food and drink, and supplements, and
the estimation module has a function of evaluating an effect of ingesta on dementia.

32. The system according to claim 28,
wherein the estimation module has a function of estimating an affection state of a first causative disease.

33. The system according to claim 32,
wherein the first input module includes a configuration for acquiring the first evaluation index for differentiation of the first causative disease,
the second input module includes a configuration for acquiring the second evaluation index for differentiation of the first causative disease, and
the estimation module includes the first evaluation function for estimating the affection state of the first causative disease.

34. The system according to claim 33,
wherein the first input module includes a configuration for acquiring, as the first evaluation index, at least one of following values:
a: output softmax value of an activation function when estimating the first causative disease by a deep learning differentiation model using a brain image as its input,
b: output softmax value of an activation function when estimating the first causative disease by the deep learning differentiation model further using a filtered image of a brain image as its input in a region of interest obtained by statistical processing of brain images,
c: volume value or blood flow rate of a region of interest by statistical processing of brain images,
d: Z-score value of volume or blood flow evaluation of a region of interest by statistical processing of brain images,
e: volume value or blood flow rate of a region of interest when estimating the first causative disease by the deep learning differentiation model using a brain image as its input,
f: Z-score value of volume or blood flow evaluation of a region of interest when estimating the first causative disease by the deep learning differentiation model using a brain image as its input.

35. The system according to claim 33,
wherein the second input module includes a configuration for acquiring the second evaluation index including a result of a cognitive test suitable for differentiation of the first causative disease.

36. The system according to claim 33,
wherein the estimation module includes the first evaluation function that estimates the first causative disease when the evaluation value exceeds a first threshold value.

37. The system according to claim 33,
wherein the first evaluation index is odds x1 of the first causative disease, the second evaluation index is odds x2 of the first causative disease, and the estimation module includes a configuration for calculating the evaluation value s by a following first evaluation function,
s=x1×x2.

38. The system according to claim 33,
wherein, assuming that the first evaluation index and the second evaluation index are xi, the estimation module includes a configuration for calculating an affection probability py* of a causative disease y* as the evaluation value by using a following first evaluation function,
$$\begin{matrix}[ Equation 1 ] & \\ {p}_{y *} = \frac{exp ( - {{\sum }_{i}}^{n} {w}_{y * i} {x}_{i} )}{{\sum }_{y ∈ Y} exp ( - {{\sum }_{i}}^{n} {w}_{yi} {x}_{i} )} & \end{matrix}$$
where Y is a set of all diseases to be evaluated, wyi is a weighting factor of each evaluation index of each causative disease, and i is an integer.

39. The system according to claim 28,
wherein the estimation module includes a configuration for providing information as a stratification marker.

40. The system according to claim 28, further comprising:
a module that outputs an estimation of the estimation module and/or a background to the estimation, the first evaluation index, the second evaluation index, and other pieces of information to output media including a smartphone, a PC, a tablet, and paper.

41. The system according to claim 28, further comprising:
a module that classifies subjects into predetermined categories based on an estimation of the estimation module and/or a background to the estimation, the first evaluation index, the second evaluation index, and other pieces of information.

42. A system, comprising:
a calculation unit that calculates a region evaluation value for each of a plurality of regions of interest of a brain of a subject based on a first evaluation index for each of the plurality of regions of interest and a weighting factor corresponding to each first evaluation index; and
an output unit that outputs each region evaluation value calculated by the calculation unit.

43. The system according to claim 42,
wherein the output unit outputs display data for displaying region evaluation values of the plurality of regions of interest of the subject's brain so as to be arranged in a predetermined order.

44. The system according to claim 42, further comprising:
a reception unit that receives a selection of a required subject from a plurality of subjects,
wherein the calculation unit calculates a region evaluation value for each of the plurality of regions of interest of a brain of the selected subject.

45. The system according to claim 42, further comprising:
a healthy person evaluation value acquisition unit that acquires a region evaluation value for each of a plurality of regions of interest of a healthy person,
wherein the output unit outputs display data for displaying region evaluation values for a plurality of regions of interest of the subject and healthy subjects in a display mode that allows comparison.

46. The system according to claim 42, further comprising:
a brain disease patient evaluation value acquisition unit that acquires a region evaluation value for each of a plurality of regions of interest relevant to a required brain disease of a brain disease patient,
wherein the output unit outputs display data for displaying region evaluation values for a plurality of regions of interest of the subject and brain disease patients in a display mode that allows comparison.

47. The system according to claim 42, further comprising:
an estimation unit that estimates a state of a brain disorder, including dementia, of the subject based on a whole-brain evaluation value obtained by a second evaluation function whose variable is the first evaluation index for each of the plurality of regions of interest of the subject's brain.

48. The system according to claim 47,
wherein the second evaluation function is expressed by a linear combination of the first evaluation index with the weighting factor corresponding to each first evaluation index as a coefficient, which is predicted by ridge regression using learning data.

49. The system according to claim 47, further comprising:
a whole-brain evaluation value acquisition unit that acquires a whole-brain evaluation value of a healthy person and a whole-brain evaluation value relevant to a required brain disease of a brain disease patient,
wherein the output unit outputs display data for displaying a whole-brain evaluation value of the subject and the whole-brain evaluation values of the healthy person and the brain disease patient in a display mode that allows comparison.

50. The system according to claim 42,
wherein the first evaluation index includes a Z-score value of a gray matter volume value of a region of interest on an anatomical standard space, a blood flow rate in a region of interest, or an amount of accumulation of malignant proteins in a region of interest.

51. A method for controlling a system including a first input module configured to acquire a first evaluation index based on data regarding a physical state of a brain of a subject, a second input module configured to acquire a second evaluation index based on data regarding a function of the brain of the subject, and an estimation module configured to estimate a state of dementia of the subject, the control method comprising:
acquiring the first evaluation index and the second evaluation index through the first input module and the second input module by the estimation module; and
estimating a state of a brain disorder, including dementia, of the subject based on an evaluation value obtained by a first evaluation function having the first evaluation index and the second evaluation index as its variables.

52. An information providing method, comprising:
calculating a region evaluation value for each of a plurality of regions of interest of a brain of a subject based on a first evaluation index for each of the plurality of regions of interest and a weighting factor corresponding to each first evaluation index; and
outputting each calculated region evaluation value.

53. A computer readable non-transitory recording medium recording a computer program having instructions for causing a computer to execute:
acquiring a first evaluation index based on data regarding a physical state of a brain of a subject;
acquiring a second evaluation index based on data regarding a function of the brain of the subject; and
estimating a state of a brain disorder, including dementia, of the subject based on an evaluation value obtained by a first evaluation function having the first evaluation index and the second evaluation index as its variables.

54. A computer readable non-transitory recording medium recording a computer program causing a computer to execute processing for:
calculating a region evaluation value for each of a plurality of regions of interest of a brain of a subject based on a first evaluation index for each of the plurality of regions of interest and a weighting factor corresponding to each first evaluation index; and
outputting each calculated region evaluation value.
